(19) United States
(12) Patent Application Publication
(10) Pub. No.: US 2024/0000985 A1
(43) Pub. Date: Jan. 4, 2024

(54) INDUCTIVE HEAT STERILIZING SYSTEMS, DEVICES, AND METHODS

(71) Applicant: Genentech, Inc., South San Francisco, CA (US)

(72) Inventors: Luis Fernando Caldera, El Cerrito, CA (US); Arthi Narayanan, Cupertino, CA (US); Trishna Ray-Chaudhuri, Pleasant Hill, CA (US); Nicholas Rummel, San Francisco, CA (US)

(73) Assignee: Genentech, Inc., South San Francisco, CA (US)

(21) Appl. No.: 18/204,281

(22) Filed: May 31, 2023

Related U.S. Application Data
(63) Continuation of application No. PCT/US2021/061278, filed on Nov. 30, 2021.
(60) Provisional application No. 63/119,896, filed on Dec. 1, 2020.

(51) Int. Cl. A61L 2/04; H05B 6/10; A61L 2/26; A61L 2/24

(52) U.S. Cl. CPC A61L 2/04; H05B 6/105; A61L 2/26; A61L 2/24; A61L 2202/14

(57) ABSTRACT

A system for making a sterile connection of at least one conduit is provided. The system includes a clamp for securing the conduit and for delivering inductive energy to sterilize an interior of the conduit. The system includes an insert for delivering inductive energy to sterilize an interior of the conduit. The system includes a cradle for directly supporting the clamp and indirectly supporting the insert inside the conduit. The system includes an operating system and a device for generating inductive heat. Related methods, apparatuses, devices, systems, techniques and articles are also described.

### CROSS-REFERENCE TO RELATED APPLICATIONS

This application is a continuation of International Application No. PCT/US2021/061278 having an International filing date of Nov. 30, 2021, which claims benefit of priority to U.S. Provisional Application No. 63/119,896, filed on Dec. 1, 2020, the contents of which are incorporated herein by reference in their entirety.

### FIELD

The present disclosure relates to a system, device and method for making a sterile connection of at least one conduit and an insert. Specifically, the present disclosure relates to an insert comprising a body having an inner surface and an outer surface, the body configured to connect with a conduit, which may include one or more sections of elastomeric tubes, e.g., silicone tubes. A system including the insert and an apparatus (or cradle) for holding the insert and the elastomeric tubes is also described. An inductive heating device for providing energy to the system is also described. A computer system for controlling the system is further provided. Related apparatuses, systems, techniques and articles are also described.

### BACKGROUND

Developed devices and methods for making a sterile connection of at least one conduit involved aseptic connectors. A system for researching, developing and manufacturing treatments suspended in liquid media may involve more than 100 connections. Prior systems used a combination of welding and aseptic connectors. Welding is not suited for silicone tubing. Aseptic connectors are relatively expensive, have limited shelf life, must be incorporated into drawings, and can have complicated connection procedures associated. Silicone tubing is preferred because they withstand relatively high flow rates, relatively high pressures, pumping operations, and are manufactured in an animal-free manner.

Such developed devices and methods did not accommodate relatively larger tubing sizes, did not reliably achieve sterilization conditions across critical points within the connection, took too long to sterilize, took too long to cool down to an acceptable temperature, had insufficient means for monitoring temperature at critical points within this system, and failed tests for sterility, air leaks, tensile strength, and the like.

The present inventors developed improvements of devices and methods in making a sterile connection of at least one conduit that overcome at least the above-referenced problems with the devices and methods of the related art.

### SUMMARY

One or more of the following features may be included in any feasible combination.

An insert for connecting a conduit having an outer surface and an inner surface is provided. The insert may include a body having an inner surface and an outer surface, the body configured to connect with the conduit having the outer surface and the inner surface. The outer surface of the body of the insert may be configured to engage with the inner surface of the conduit. The body may be configured for inductive heating of a connection of the insert and the conduit to a sterilizing temperature. The body may be configured for inductive heating for a duration of time. An interior of the connection of the insert and the conduit may be sterilized as a result of the inductive heating.

The sterilizing temperature may be between about 160° C. (about 320° F.) and about 350° C. (about 662° F.), and the duration of time may be between about 60 seconds and about 300 seconds.

The body may be configured for inductive heating for the duration of time of about 100 seconds.

The conduit may have an outer diameter of between about 0.125 inches (about centimeter) and about 1.25 inches (about 3.175 centimeters).

The outer surface of the body may include at least one rib. The at least one rib may include 3 ribs at each end of the insert. Each of the 3 ribs may include a radially extending ridge forming an engagement surface configured for engagement with the inner surface of the conduit.

A system comprising the insert and a clamp configured to transmit inductive heat to the conduit is provided. The clamp may include carbon steel. The clamp may include a base and a collar extending beyond the base. The collar may be configured to make contact with a portion of the conduit in a critical zone located between a clamp zone directly adjacent to the clamp and an insulated zone formed by an overlap of the insert and the conduit. The collar may have a frustoconical shape. The collar may have an inwardly facing surface configured to substantially conform with an outer surface of the clamped conduit.

The system may include an apparatus configured to make contact with the insert and the clamp and to deliver energy from an external energy source to the insert and the clamp in order to generate inductive heat in the insert and the clamp.

The system may include an apparatus configured to accommodate or hold the conduit, the insert, and the clamp during the generation of the inductive heat.

The system may include an induction controller configured to control provision of energy to the apparatus to heat the insert and the clamp, which transfer heat to sterilize the conduit.

A method for connecting a conduit with an insert is provided. The method may include clamping a conduit with a clamp. The method may include cutting a portion of the conduit leaving an extending portion. The method may include inserting an end of the insert into the extending portion. The method may include securing the extending portion to the insert with a compression device. The method may include electrically connecting an inductive heating device to the insert. The method may include heating the insert with energy from the inductive heating device. The method may include maintaining a predetermined temperature for a duration of time in order to form a sterile connection of an interior of the conduit and the insert. The method may include soaking the sterile connection. The method may include cooling the sterile connection. The method may include removing the clamp from the sterile connection. The clamp may be a collared clamp. The predetermined temperature may be between about 160° C. (about 320° F.) and about 350° C. (about 662° F.). The duration of time may be between about 60 seconds and about 300 seconds.

Electrically connecting the inductive heating device to the insert may include electrically connecting the inductive heating device to the clamp. Heating the insert with energy from the inductive heating device may include heating the clamp with energy from the inductive heating device.

The method may involve an apparatus configured to accommodate or hold the conduit, the insert, and the clamp during the generation of the inductive heat. The apparatus may be formed of a conductive material, which conducts the inductive energy from the inductive heating device to at least the insert and the clamp.

A method of connecting a conduit with an insert is provided. A device may be provided, the device having at least one processor and a memory storing at least one program for execution by the at least one processor. The at least one program may include instructions, which, when executed by the at least one processor cause the at least one processor to perform operations. The operations may include transmitting instructions to an inductive heating device to transmit heat energy to the insert from the inductive heating device. The operations may include determining a temperature of at least one sensor within the conduit and/or the insert. The operations may include maintaining the determined temperature equal to or above a predetermined temperature for a duration of time in order to form a sterile connection of an interior of the conduit and the insert. The predetermined temperature may be between about 160° C. (about 320° F.) and about 350° C. (about 662° F.).

A system for connecting a conduit with an insert is provided. The system may include a device having at least one processor and a memory storing at least one program for execution by the at least one processor. The at least one program may include instructions, when, executed by the at least one processor cause the at least one processor to perform operations. The operations may include transmitting instructions to an inductive heating device to transmit heat energy to the insert from the inductive heating device. The operations may include determining a temperature of at least one sensor within the conduit and/or the insert. The operations may include maintaining the determined temperature equal to or above a predetermined temperature for a duration of time in order to form a sterile connection of an interior of the conduit and the insert. The predetermined temperature may be between about 160° C. (about 320° F.) and about 350° C. (about 662° F.).

A non-transitory computer-readable storage medium storing at least one program for connecting a conduit with an insert is provided. The at least one program may be for execution by at least one processor and a memory storing the at least one program. The at least one program may include instructions, when, executed by the at least one processor cause the at least one processor to perform operations. The operations may include transmitting instructions to an inductive heating device to transmit heat energy to the insert from the inductive heating device. The operations may include determining a temperature of at least one sensor within the conduit and/or the insert. The operations may include maintaining the determined temperature equal to or above a predetermined temperature for a duration of time in order to form a sterile connection of an interior of the conduit and the insert. The predetermined temperature may be between about 160° C. (about 320° F.) and about 350° C. (about 662° F.).

A clamp for transmitting inductive heat to a conduit is provided. The clamp may include carbon steel. The clamp may include a base and a collar extending beyond the base. The collar may be configured to make contact with a portion of the conduit in a critical zone located between a clamp zone directly adjacent to a clamp attached to the conduit and an insulated zone formed by an overlap of an insert and the conduit. The collar may have a frustoconical shape. The collar may have an inwardly facing surface configured to substantially conform with an outer surface of the clamped conduit. The clamp and the collar may be configured to promote development of a predetermined temperature of an interior of insert and the conduit. The predetermined temperature may be between about 160° C. (about 320° F.) and about 350° C. (about 662° F.).

An apparatus for indirectly supporting an insert and making direct contact with a clamp and for delivering energy from an external energy source to the insert and the clamp in order to generate inductive heat in the insert and the clamp is provided. The apparatus may include conductive material. The apparatus may include a main groove in a top of a body of the apparatus. The apparatus may include at least one peripheral block. The apparatus may include a central groove in the at least one peripheral block. The central groove may be aligned with the main groove. The apparatus may include at least one central block. A peripheral groove may be formed between the at least one peripheral block and the at least one central block. The apparatus may be configured to concentrate inductive heat within one or more of the main groove, the central groove and/or the peripheral groove. The apparatus may be configured to promote development of a predetermined temperature of an interior of the insert, the clamp, and the conduit, the predetermined temperature being between about 160° C. (about 320° F.) and about 350° C. (about 662° F.). The apparatus may be further configured to accommodate or hold a conduit, the insert, and the clamp during the generation of the inductive heat. The apparatus may include an induction controller configured to control provision of energy to the apparatus to heat the insert and the clamp, which transfer heat to sterilize the conduit.

An apparatus for indirectly supporting an insert and making direct contact with a clamp and for positioning the insert and the clamp so that energy may be delivered from an external energy source to the insert and the clamp in order to generate inductive heat in the insert and the clamp is provided. The apparatus may include a conduit support surface. The apparatus may include an accommodating space for accommodating a conduit, the insert and the clamp. The apparatus may include a first clamp support surface. The apparatus may include a second clamp support surface. The apparatus may be configured to concentrate inductive heat within the accommodating space.

The apparatus may be configured to promote development of a predetermined temperature of an interior of the insert, the clamp, and the conduit, the predetermined temperature being between about 160° C. (about 320° F.) and about 350° C. (about 662° F.).

The apparatus may be further configured to accommodate or hold a conduit, the insert, and the clamp during the generation of the inductive heat.

The apparatus may include a pair of end plates, each of the pair of end plates including the conduit support surface. The conduit support surface may include a U-shaped support surface.

The conduit support surface may include a pair of conduit support surfaces. The accommodating space may be provided between the pair of conduit support surfaces.

The apparatus may include a pair of support bars. Each of the pair of support bars may include the first clamp support surface.

The first clamp support surface may include a groove having a central axis substantially perpendicular to a central axis of the accommodating space.

The first clamp support surface may include a groove and a projection. The groove and the projection may have clamp engagement surfaces, which are substantially perpendicular to each other.

The first clamp support surface may include a chamfer.

The first clamp support surface may be configured to make direct contact with pivoted conduit clamping jaws of the clamp.

The apparatus may include an inverse-U-shaped bracket. The inverse-U-shaped bracket may include the second clamp support surface.

The second clamp support surface may include a curved surface and linear surfaces on either side of the curved surface.

The second clamp support surface may be configured to make direct contact with at least one arm of the clamp.

A clamp for indirectly supporting an insert and for making direct contact with a clamp and for delivering energy from an external energy source to the insert and the clamp in order to generate inductive heat in the insert and the clamp is provided. The clamp may include a collar including conduit engagement surfaces. The conduit engagement surfaces may include a posterior transition surface and a secondary posterior engagement surface. The posterior transition surface may be a tapered and curved arc providing a smooth transition from a curved surface of the posterior transition surface to a relatively flat surface of the secondary posterior engagement surface. The conduit engagement surfaces may include a pair of secondary posterior transition surfaces on either side of the primary posterior engagement surface. Each of the pair of secondary posterior transition surfaces may have a triangular arced shape configured to provide a smooth transition from the curved surface of the primary posterior engagement surface and the relatively flat surface of the secondary posterior engagement surface.

These and other capabilities of the disclosed subject matter will be more fully understood after a review of the following figures, detailed description, and claims.

### DESCRIPTION OF DRAWINGS

These and other features will be more readily understood from the following detailed description taken in conjunction with the accompanying drawings, in which:

FIG. 1 is a perspective view of a first system for making a sterile connection between at least one conduit and an insert including an induction heating device and a first cradle according to an exemplary embodiment;

FIG. 2 is a close-up view of a control panel of the first system according to an exemplary embodiment;

FIG. 3 is a perspective view of a second system for making a sterile connection between at least one conduit and an insert including an induction heating device, a second cradle and a protective cover (in a closed position) according to an exemplary embodiment;

FIG. 4 is a plan view of a sterile connection of two conduits and an insert according to an exemplary embodiment;

FIG. 5 is a perspective view of the second system with the protective cover in an open position, with two conduits and an insert held in the second cradle, and with two clamps, one for each conduit according to an exemplary embodiment;

FIG. 6 is a schematic diagram of a sterile connection with clamps, the sterile connection being made between a media bag and an external piece of equipment (not shown) according to an exemplary embodiment;

FIG. 7 is a plan view of a first insert and a second insert according to exemplary embodiments;

FIG. 8 is a perspective view of an end of a third insert according to an exemplary embodiment;

FIG. 9 is a plan view of a conduit engaged with an insert and held with a first clamp according to an exemplary embodiment;

FIG. 10 is a plan view of a conduit engaged with an insert and held with a second clamp according to an exemplary embodiment;

FIG. 11 is a side view of the first clamp and the second clamp according to exemplary embodiments;

FIG. 12 is a schematic representation of an insert-only configuration including a temperature profile through a longitudinal cross-section of the insert-only configuration, i.e., a conduit engaged with an insert and without any clamp according to an exemplary embodiment;

FIG. 13 is a schematic representation of an insert-plus-clamp configuration including a temperature profile through a longitudinal cross-section of the insert-plus-clamp configuration, i.e., a conduit engaged with an insert and a first clamp according to an exemplary embodiment;

FIG. 14 is a schematic representation of an insert-plus-collared-clamp configuration including a temperature profile through a longitudinal cross-section of the insert-plus-collared-clamp configuration, i.e., a conduit engaged with an insert and a second clamp according to an exemplary embodiment;

FIG. 15 is similar to FIG. 9 and annotated with demarcations signifying four zones of interest according to an exemplary embodiment;

FIG. 16A is a plot of temperature over time for the insert-plus-clamp configuration of FIG. 15 with thermocouples placed at each of the four zones depicted in FIG. 15, including two readings within Zone D, and a “kill zone” temperature according to an exemplary embodiment;

FIG. 16B is a plot of temperature over time in a “critical zone” or “kill zone” of the insert-only configuration of FIG. 12, the insert-plus-clamp configuration of FIG. 13, and the insert-plus-collared-clamp configuration of FIG. 14 according to exemplary embodiments;

FIG. 17 is a perspective view of the second system connected to an external device and a sterile connection of two media bags formed by the second system according to an exemplary embodiment;

FIG. 18 is a perspective view of a sterile connection of two media bags with additional conduits according to an exemplary embodiment;

FIG. 19 is a perspective view of a sterile connection of a media bag with relatively large diameter, polymer reinforced conduits (as well as the second cradle) according to an exemplary embodiment;

FIG. 20 is a perspective view of a pressure testing system connected to the connections shown in FIG. 21 according to an exemplary embodiment;

FIG. 21 is a perspective view of four sterile connections having four different conduit sizes submerged in a liquid bath and connected to the pressure testing system of FIG. 20 undergoing a pressure test according to an exemplary embodiment;

FIG. 22 is a perspective view of a tensile strength test of a sterile connection according to an exemplary embodiment;

FIG. 23 is a flow chart of a process for making a sterile connection between at least one conduit and an insert according to an exemplary embodiment;

FIG. 24 is a schematic diagram of a computer device or system including at least one processor and a memory storing at least one program for execution by the at least one processor according to an exemplary embodiment;

FIG. 25A is a plot of a number of microbial survivors (log scale) versus time according to an exemplary embodiment;

FIG. 25B is a plot of D-Value (log scale) versus temperature according to an exemplary embodiment;

FIG. 26 is a plot of temperature versus time to kill 10⁶ spores according to an exemplary embodiment;

FIG. 27 is a side view of a fourth insert according to an exemplary embodiment;

FIG. 28 is an end view of the fourth insert according to an exemplary embodiment;

FIG. 29 is a side view of a fifth insert according to an exemplary embodiment;

FIG. 30 is an end view of the fifth insert according to an exemplary embodiment;

FIG. 31 is a side view of a sixth insert according to an exemplary embodiment;

FIG. 32 is an end view of the sixth insert according to an exemplary embodiment;

FIG. 33 is a first perspective view of a third system for making a sterile connection between at least one conduit and an insert including an induction heating device, a third cradle, and a pair of third clamps according to an exemplary embodiment;

FIG. 34 is a second perspective view of the third system according to an exemplary embodiment;

FIG. 35 is a rear perspective view of the third system according to an exemplary embodiment;

FIG. 36A is a perspective view of a main housing of the induction heating device of the third system according to an exemplary embodiment;

FIG. 36B is an exploded perspective view of the main housing of the induction heating device of the third system according to an exemplary embodiment;

FIG. 36C is an exploded perspective view of the third cradle, the conduits, the insert, inductive heating coils, a laser for temperature measurement, thermocouples, cooling fans, a protective shield, and a base platform of the third system according to an exemplary embodiment;

FIG. 37 is a lateral-facing side perspective view of a left-hand-side one of the pair of third clamps in a closed position according to an exemplary embodiment;

FIG. 38 is a medial-facing side perspective view of the left-hand-side one of the pair of third clamps in the closed position according to an exemplary embodiment;

FIG. 39 is an anterior side view of the left-hand-side one of the pair of third clamps in the closed position according to an exemplary embodiment;

FIG. 40 is an exploded medial-facing side perspective view of the left-hand-side one of the pair of third clamps in the closed position according to an exemplary embodiment;

FIG. 41 is a bottom side view of the left-hand-side one of the pair of third clamps in the closed position according to an exemplary embodiment;

FIG. 42 is a medial side view of the left-hand-side one of the pair of third clamps in the closed position according to an exemplary embodiment;

FIG. 43 is a lateral side view of the left-hand-side one of the pair of third clamps in the closed position according to an exemplary embodiment;

FIG. 44 is a medial-facing side perspective view of a right-hand-side one of the pair of third clamps in a closed position according to an exemplary embodiment;

FIG. 45 is a lateral side view of the right-hand-side one of the pair of third clamps in the closed position according to an exemplary embodiment;

FIG. 46 is a posterior side view of the right-hand-side one of the pair of third clamps in the closed position according to an exemplary embodiment;

FIG. 47 is an anterior side view of the right-hand-side one of the pair of third clamps in the closed position according to an exemplary embodiment;

FIG. 48 is a medial side view of the right-hand-side one of the pair of third clamps in the closed position according to an exemplary embodiment;

FIG. 49 is a top side view of the right-hand-side one of the pair of third clamps in the closed position according to an exemplary embodiment;

FIG. 50 is a bottom side view of the right-hand-side one of the pair of third clamps in the closed position according to an exemplary embodiment;

FIG. 51 is a front perspective view of the third system for making a sterile connection between at least one conduit and an insert including the induction heating device, the third cradle, and the pair of third clamps according to an exemplary embodiment;

FIG. 52 is a perspective view of the pair of third clamps engaged with a pair of conduits, which are connected with an insert according to an exemplary embodiment;

FIG. 53 is an anterior perspective view of the third cradle according to an exemplary embodiment;

FIG. 53 is a posterior perspective view of the third cradle according to an exemplary embodiment;

FIG. 54 is an anterior perspective view of the third cradle according to an exemplary embodiment;

FIG. 55 is a posterior perspective view of the third cradle engaged with the pair of conduits and the pair of third clamps (also, the insert between the pair of conduits) according to an exemplary embodiment;

FIG. 56 is an anterior perspective view of the third cradle engaged with the pair of conduits and the pair of third clamps (also, the insert between the pair of conduits) according to an exemplary embodiment;

FIG. 57 is a posterior side view of the third cradle including spring loaded bolts and hex nuts according to an exemplary embodiment;

FIG. 58 is a right-hand side view of the third cradle including spring loaded bolts and hex nuts according to an exemplary embodiment;

FIG. 59 is a left-hand side view of the third cradle including spring loaded bolts and hex nuts according to an exemplary embodiment;

FIG. 60 is an anterior side view of the third cradle including spring loaded bolts and hex nuts according to an exemplary embodiment;

FIG. 61 is a top side view of the third cradle including spring loaded bolts and hex nuts according to an exemplary embodiment;

FIG. 62 is a bottom side view of the third cradle including spring loaded bolts and hex nuts according to an exemplary embodiment; and

FIG. 63 is a posterior perspective view of the third cradle including spring loaded bolts and hex nuts according to an exemplary embodiment.

It is noted that the drawings are not necessarily to scale. The drawings are intended to depict aspects of the subject matter disclosed herein, and therefore should not be considered as limiting the scope of the disclosure. Those skilled in the art will understand that the structures, systems, devices, and methods specifically described herein and illustrated in the accompanying drawings are non-limiting exemplary embodiments and that the scope of the present invention is defined solely by the claims.

### DETAILED DESCRIPTION

The cost of producing biologic pharmaceutical treatments, such as monoclonal antibody therapies, may be on the order of $100,000 per year. As these costs have increased, a need for versatile, sterile and reliable connections of components of systems for conveying fluids in laboratory conditions, including connections of media bags via silicone tubing, connections of one piece of silicone tubing to another, and the like, has also increased. As noted above, developed technologies have numerous problems. The present systems, devices and methods consistently overcome the problems of the developed technologies. While various exemplary systems are described, each of the exemplary systems, including the first exemplary system, the second exemplary system, the third exemplary system, the insert or needle, the process and control methods, the computer-implemented control methods, and/or the like, may include one or more components that may be used with and/or interchanged with one or more components of another exemplary system (e.g., the first exemplary system, the second exemplary system, the third exemplary system, the insert or needle, the process and control methods, the computer-implemented control methods, and/or the like).

First Exemplary System

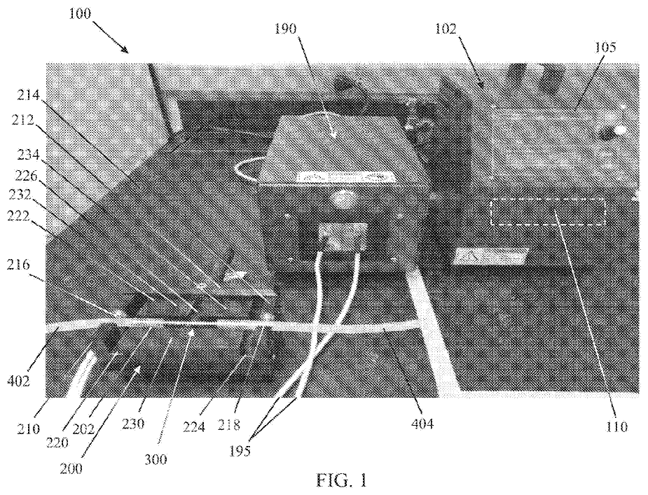

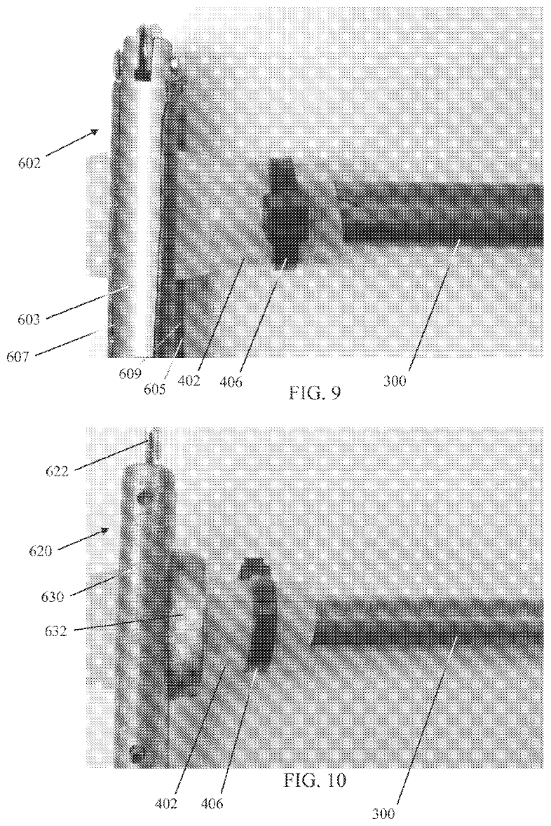

An exemplary embodiment of a system for sterilization using inductive heating is shown in FIG. 1. A system (e.g., 100) including an insert (e.g., 300; which may also be referred to as a “needle”), conduits (e.g., 402, 404), a cradle (e.g., 200) for holding the insert and the conduits, a clamp (e.g., 602, 620; see, FIGS. 9 and 10), and an inductive heating device (e.g., 102) may be provided for sterilizing a connection (e.g., 499) between two of the conduits 402, 404 via at least the insert 300 either alone or in combination with another structure such as the clamp 602. Each component of the system 100 may be configured to achieve sterilization by heating the connection 499 to at least about 160° C. (about 320° F.); holding the heated condition for at least about 100 seconds up to about 10 minutes; and, after sterilization, cooling down one or more components of the system 100 to a desired temperature in about 100 seconds. Each of the components of the system 100 may result in cost savings and reduction of waste due to incomplete sterilization, improper connections, leaks, and the like.

Although achieving a temperature of at least about 160° C. (about 320° F.) and holding the heated condition for at least about 100 seconds is desirable, it is further desirable in some exemplary embodiments to avoid exceeding a temperature of at least about 450° C. (about 842° F.), which is a glass transition temperature for a typical silicon tube. In some exemplary embodiments, it is desirable to avoid exceeding a temperature of at least about 400° C. (about 752° F.). In some exemplary embodiments, it is desirable to avoid exceeding a temperature of at least about 350° C. (about 662° F.), e.g., about 343° C. (about 649.4° F.), to avoid undesirable deformation of conduits, particularly silicone tubing. The temperature may be any value or subrange within the recited ranges, including endpoints.

The system 100 may include a controller (e.g., 110) configured to execute instructions for operation of the components of the system 100 to achieve sterilization. The controller 110 may be configured to control supply of inductive heat to other components of the system 100. The controller 110 may be configured to operate the inductive heating device 102 to provide inductive heat to one or more of the cradle 200, the insert 300 and/or clamps 602, 620. The controller 110 may be configured to display output on a control panel 105 and receive input from sensors, dials (e.g., 110) and push buttons (e.g., 115, 120). In other embodiments, a touch screen may be used in combination with buttons and dials (see, e.g., FIGS. 33-36C and 51, hereinbelow). The controller 110 may be configured to output temperature readings, time durations, power measurements, percentages, status messages, program numbers, recipe numbers, warnings, confirmations, and the like.

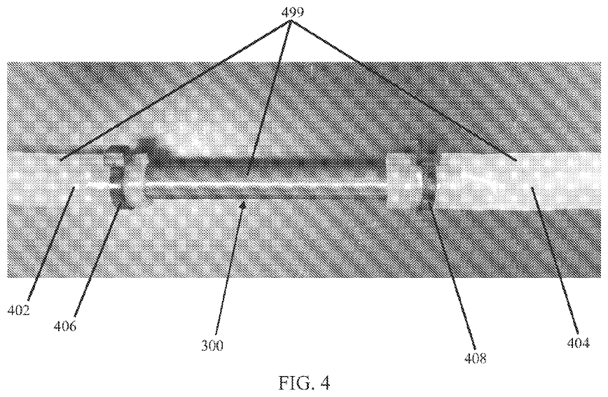

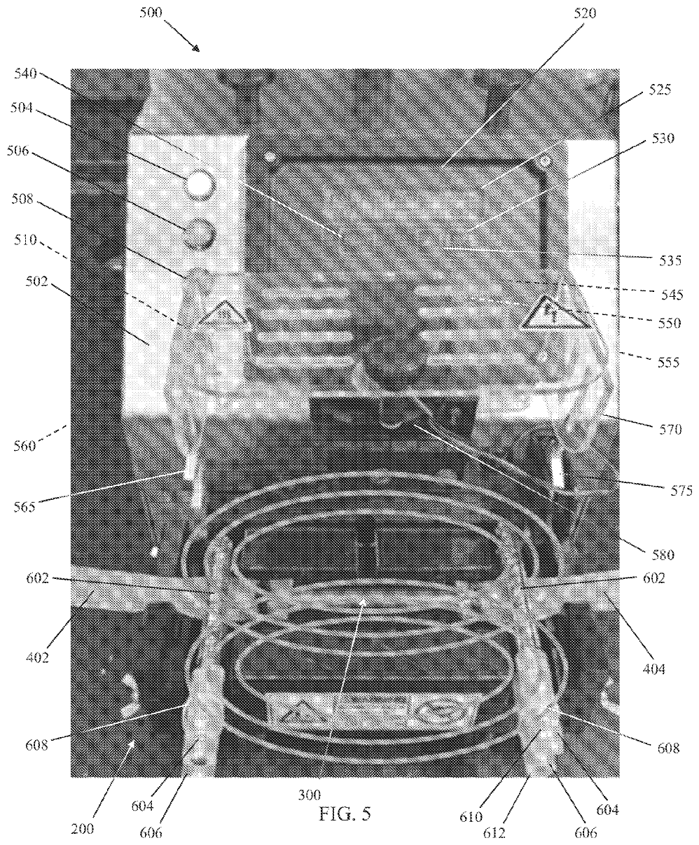

The inductive heating device 102 may be operatively connected to a separate enclosure 190 including power outputs connected to wires 195 connected to supply inductive power to the cradle 200. The inductive heating device 102 may include a wire. When electric current flows through the wire, a magnetic field may be created. When a direction of the current is reversed, the magnetic field may be reversed. The inductive heating device 102 may include an inductive coil, which may generate an electromagnetic field (e.g., depicted with two sets of partially overlapping concentric ovals in FIG. 5), which may induce a current in a conductive material within the electromagnetic field.

The cradle 200 may be made of conductive material. The cradle 200 may be configured to hold the conduit 402, 404 and the insert 300 in a main groove 202 in a top of a body of the cradle 200. The cradle 200 may include one or more blocks; in some embodiments, the cradle 200 includes a first peripheral block 210, a second peripheral block 212, and a third peripheral block 214. Top surfaces of the first, second and third peripheral blocks 210, 212, 214 may form a continuous C-shape when viewed from above. Each of the first and third peripheral blocks 210, 214 may include a central groove 216, 218, respectively, aligned with the main groove 202. The central grooves 216, 218 may include detents for holding the conduits 402, 404. Each of the central grooves 216, 218 may have a size configured for a particular size of tubing. The central grooves 216, 218 may be fitted with removable pieces configured for each of, for example, conduits 402, 404 having a diameter of between about 0.125 inches (about 0.3175 centimeter) and about 1.250 inches (about 3.175 centimeters), including about 0.250 inches (about 0.635 centimeter), about 0.500 inches (about 1.27 centimeters), about 0.750 inches (about 1.905 centimeters), and about 1.000 inches (about 2.54 centimeters). In some embodiments, the central grooves 216, 218 may have a circular shape to match the circular shape of the conduits 402, 404.

A central portion of the top of the cradle 200 may include one or more blocks; in some embodiments, the central portion of the top of the cradle 200 has a first central block 230, a second central block 232, and a third central block 234. A first peripheral groove 220 may be formed between ends of the first and second central blocks 230, 232 and the first peripheral block 210. The first peripheral groove 220 may be approximately perpendicular to the main groove 202. A second peripheral groove 222 may be formed between ends of the second and third central blocks 232, 234 and the second peripheral block 212. The second peripheral groove 222 may be approximately parallel to the main groove 202. A third peripheral groove 224 may be formed between opposite ends of the first and third central blocks 230, 234 and the third peripheral block 214. The third peripheral groove 224 may be approximately perpendicular to the main groove 202. A secondary groove 226 may separate the second central block 232 from the third central block 234. The secondary groove 226 may be approximately perpendicular to the main groove 202. The cradle 200 may be configured to concentrate inductive heat within one or more of the aforementioned grooves. In some exemplary embodiments, the main groove 202, the first peripheral groove 220, and the third peripheral groove 224 are configured to concentrate inductive heat within the grooves 202, 220 and 224. The grooves 202, 220 and 224 may be provided within the electromagnetic field generated by the cradle 200 connected to the inductive heating device 102. In some exemplary embodiments, clamps 602, 620 made of conductive material are placed within the grooves 220 and 224, which are within the electromagnetic field; thus, the clamps 602, 620 may be heated by inductive heating along with the insert 300.

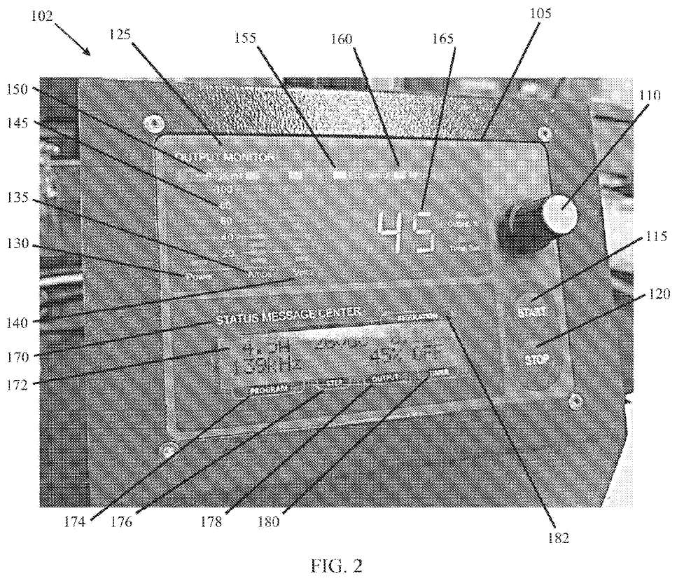

In the exemplary embodiments of FIGS. 1 and 2, the control panel 105 of the inductive heating device 102 may include a rotary dial 110 for changing settings, a start button 115 for starting the system 100, a stop button 120 for stopping the system 100, an output monitor 125 displaying information about the system 100, and a status message center 170 displaying additional information about the system 100. The output monitor 125 may include a power monitor 130, an amperage monitor 135, and a voltage monitor 140, which may display along a percentage scale 145 between 0% and 100%. The output monitor 125 may include LEDs 150, for each of the power monitor 130, the amperage monitor 135, and the voltage monitor 140, which may light to indicate the electrical output of the heating device 102. The output monitor 125 may include an LED 155, which may light to indicate an external control mode, and an LED 160, which may light to indicate display of output in terms of electromagnetic or radio frequency (RF) voltage. The output monitor 125 may include an LED display 165 configured to display numbers associated with one or more parameters. The status message center 170 may include a display configured to display two lines of status information 172, which may include a program field 174, a step field 176, an output field 178, a timer field 180, and a regulation field 182. The status message center 170 may be configured to display a temperature, a time duration, and the like. In the exemplary embodiment of FIG. 2, the status message center 170 is operating in a mode configured to display an amperage, e.g., “4.5 A”, a voltage, e.g., “26 Vdc”, a wattage, e.g., “0.12 kW”, a frequency, e.g., “139 kHz”, an output level, e.g., “45%”, and a status of a timer, e.g., “OFF”. In a different mode than the one displayed, the status message center 170 may display a program number in the program field 174, a step number in the step field 176, and the like.

Second Exemplary System

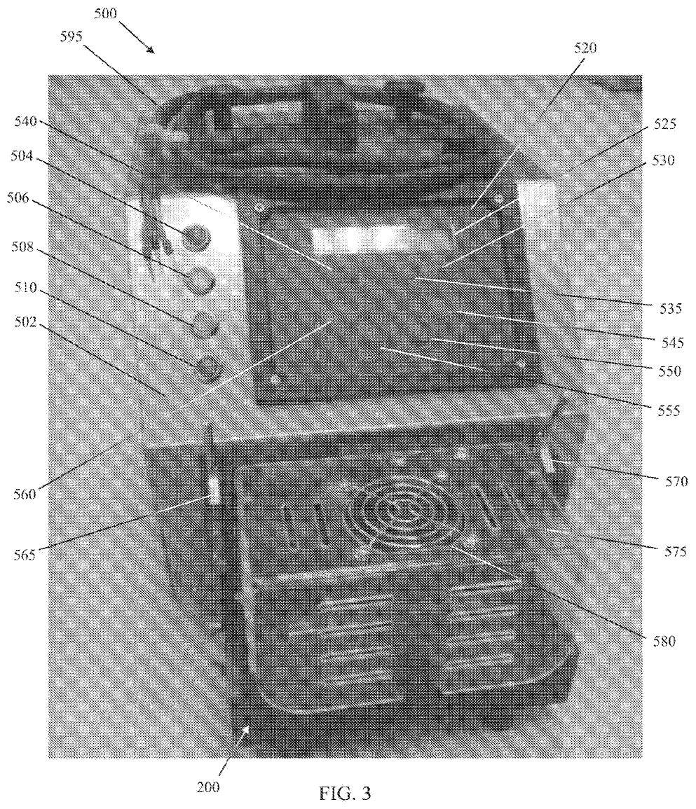

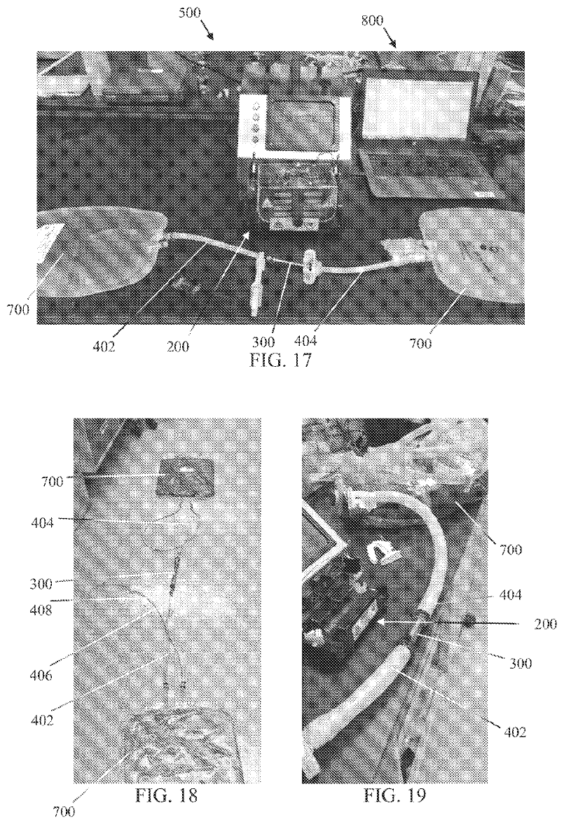

Another exemplary embodiment of a system 500 for sterilization using inductive heating is shown in FIGS. 3, 5, 17, and 19. A system 500 may include a housing of an inductive heating device 502. The cradle 200 may be attached to the housing of the inductive heating device 502 or provided separate therefrom and slid into and out of a position proximate the housing of the inductive heating device 502. Portions of the inductive heating device 502 may be contained within the housing or separate therefrom. The housing of the inductive heating device 502 may include four indicators 504, 506, 508, and 510, which may correspond with stages of the inductive heating process. In FIG. 3, the system 500 is disconnected from power, and none of the indicators 504, 506, 508, and 510 is lit. In some embodiments, the first indicator 504 may be configured to emit green light to visually indicate a “READY” state, the second indicator 506 may be configured to emit yellow light to visually indicate a “HEATING” stage, the third indicator 508 may be configured to emit blue light to visually indicate a “CYCLE COOLING” stage, and the fourth indicator 510 may be configured to emit red light to visually indicate an “ERROR” state. In FIGS. 5 and 17, the first indicator 504 is lit, which indicates that the system is ready to operate. The system 500 may include a control panel 520, which may include a digital display 525. The control panel 520 may include an up control button 530 and a down control button 535, a “SELECT” button 540, an “ALARM” indicator 545, a “STOP” button 550, a “START” button 555, and a “HEAT ON” indicator 560. The control panel 520 may incorporate features from the control panel 105 and vice versa and any suitable combination. The system 500 may include clamps 565, 570, each configured to rotate into a stowed position (as shown in FIGS. 3, 5, 17, and 19) or a deployed position (not shown) in which the clamps 565, 570 are rotated down so as to engage with the conduits 402, 404. The system 500 may include a protective cover 575 configured to enclose a space above the cradle 200. The cover 575 may include a plurality of vent holes and a handle. The cover 575 may include a cooling device 580, such as a fan, which may be operated by the control panel 520. The cooling device 580 may be configured for about 35,000 hours of maintenance free operation. The cover 575 is rotated down into a closed position in FIGS. 3 and 17; the cover 575 is rotated up into an open position in FIG. 5, and the cover 575 is removed in FIG. 19. The cover 575 is configured to prevent unintentional contact of a user with heated surfaces of the cradle 200, the insert 300, and the clamps 602, 620. The system 500 may receive electrical power via a power cord 595.

As shown, for example, in FIG. 4, the conduit 402, 404 may be an elastomeric tube. The elastomeric tube may be a silicone tube. The conduit, elastomeric tube or silicone tube 402, 404 may have a circular cross section having an outer diameter of between about 0.25 inches (about 0.635 centimeter) and about 1.25 inches (about 3.175 centimeters) and an inside diameter of between about 0.125 inches (about 0.3175 centimeter) and about 1.0 inches (about 2.54 centimeters), respectively. In some exemplary embodiments, the conduit, elastomeric tube or silicone tube 402, 404 may have a circular cross section having an outer diameter of about 0.125 inches (about 0.3175 centimeter) or less. A length of the conduit, elastomeric tube or silicone tube may be any suitable length including at least about 12 inches (about 30.48 centimeters). The diameter or length may be any value or subrange within the recited ranges, including endpoints.

In the embodiment of FIG. 5, a pair of clamps 602 are provided. Each clamp 602 may have a first arm 604 and a second arm 606. Ends of the first and second arms 604 and 606 may be inserted inside appropriately sized elastomeric sleeves 608 and 610, respectively, which may be tightened with a compression ring 608, such as a zip tie, which may be similar to compression rings 406, 408. In addition to the insert 300, each of the clamps 602 may be formed from a conductive material, which may be heated by induction from the inductive heating device 102 (or within system 500) via the cradle 200.

In some exemplary embodiments (FIGS. 9, 11, and 13), the clamp 602 may include a first arm 603 and a second arm 605. The first arm 603 may include a first conductive spacer 607 configured to engage with a first portion of the conduit 402, and the second arm 605 may include a second conductive spacer 609 configured to engage with a second portion of the conduit 402.

As demonstrated in FIG. 13, the insert 300 engages with an end of the conduit 402 to form an overlapping portion of the insert 300 and the conduit 402. Within the overlapping portion, the end of the conduit 402 closest to a center of the insert 300 is heated (via inductive heating) to a relatively high temperature compared with another end of the conduit furthest from the center of the insert 300 and closest to the clamp 602. Beyond the overlapping portion, the clamp 602 heats (via inductive heating) a portion of the conduit 402 closest to the clamp 602. However, a relatively cool section of the conduit 402 occurs between the clamp and the end of the conduit 402 closest to the center of the insert 300.

A modified version of the clamp may include additional structure. In some exemplary embodiments (FIGS. 10, 11, and 14), a collared metal clamp 620 may be provided. In some exemplary embodiments, the collared metal clamp 620 may be configured for various sizes of silicone tubing (e.g., conduits 402, 404). For example, different collared metal clamps may be provided for different sizes of silicone tubing, i.e., e.g., one for silicone tubing having an outside diameter of about 0.500 inches (about 1.27 centimeters), another for a diameter of about 0.750 inches (about 1.905 centimeters), and yet another for a diameter of about 1.000 inches (about 2.54 centimeters), and the like. A radius of inwardly facing engagement surfaces (e.g., 634, 644, see below) of the collared metal clamp 620 may be relatively larger for relatively larger tubing, and relatively smaller for relatively smaller tubing. The collared metal clamp 620 may be configured to fit directly and snugly over and in contact with the silicone tubing, like a sleeve, to maximize contact between the engagement surfaces of the collared metal clamp 620 and the silicone tubing. The collared metal clamp 620 may be made from any conductive material including carbon steel. The collared metal clamp 620 may include a first arm 630 and a second arm 640 connected to each other via a connector 622. The first and second arms 630, 640 may be configured to rotate about respective pins 624, 626 in the connector 622 to allow the arms 630, 640 to open and close about the conduit 402, 404. The first arm 630 may include a first inwardly facing engagement surface 631 configured to engage with a first portion of conduit 402, 404. The first arm 630 may include a conically shaped and curved first collar 632. The first collar 632 may include a second inwardly facing engagement surface 634 configured to engage with a second portion of conduit 402, 404. The second arm 640 may include a third inwardly facing engagement surface 641 configured to engage with a third portion of conduit 402, 404. The second arm 640 may include a conically shaped and curved second collar 642. The second collar 642 may include a fourth inwardly facing engagement surface 644 configured to engage with a fourth portion of conduit 402, 404. The arms 630, 640 may include forks 636, 646, respectively, to help hold the clamp 620 relative to the conduit 402, 404 in a desired orientation.

As demonstrated in FIG. 14, the insert 300 engages with the end of the conduit 402 to form an overlapping portion of the insert 300 and the conduit 402. Within the overlapping portion, the end of the conduit 402 closest to the center of the insert 300 is heated (via inductive heating) to a relatively high temperature. Beyond the overlapping portion, the first inwardly facing engagement surface 631 and the third inwardly facing engagement surface 641 of the collared clamp 620 heat (via inductive heating) a portion of the conduit 402 closest to the clamp 602. Unlike the clamp 602, in which the relatively cool section of the conduit 402 occurs between the clamp and the end of the conduit 402 closest to the center of the insert 300, with the collared clamp 620, the second inwardly facing engagement surface 634 and the fourth inwardly facing engagement surface 644 heat (via inductive heating) a portion of the conduit 402 between the first and third inwardly facing engagements surfaces 631, 641, and the end of the insert 300. As a result of the structure of the collared clamp 620, relative to the clamp 602, more complete heating of the connection 499 of the conduit 402 and the insert 300 is achieved. Thus, sterilization of the connection 499 is more complete.

In other words, with reference to FIGS. 13 and 15, using the needle 300 and the clamp 602, good heating via induction occurs in Zone A (the exposed portion of the needle 300), and in a portion of Zone B (a portion of the needle 300 overlapping with the conduit 402) closest to Zone A. Also, good heating via induction occurs in Zone D, which is directly adjacent to the first and second conductive spacers 607, 609 of the clamp 602. However, insufficient heating via induction occurs in Zone C, a Critical Zone, between Zones B and D. In Zone C, neither the insert 300 nor the clamp 602 are in direct contact. As seen in FIGS. 13 and 16, the temperature is significantly lower in Zone C and the left side of Zone B compared to Zone A, the right side of Zone B, and in Zone D. FIG. 16A plots a temperature (° C.) measured (via thermocouple) over time in each of the four Zones A, B, C, and D, as well as a temperature within Zone D at the clamp 602 and at the conduit 402. The target temperature of about 160° C. (about 320° F.) is denoted with a horizontal line labeled “Kill Zone”. In Zone C, the target temperature of about 160° C. (about 320° F.) is not achieved until more than about 180 seconds have passed from a start of the heating. In some modes, the conduit 402 is “soaked” at the target temperature of about 160° C. (about 320° F.) for at least about 100 seconds to ensure dry-heat sterilization of the conduit 402.

In contrast, with reference to FIG. 14, the clamp 620 achieves desirable heating at a target temperature across all four Zones A, B, C, and D in a time period that is as short as about 15 seconds and generally less than about 120 seconds. In many embodiments, desirable heating is achieved in less than about 100 seconds. In some exemplary embodiments, the system 100, 500 (including the conduit 402, the insert 300, and the clamp 620)—which may be heated via inductive heating to reach a dry heat sterilizing temperature of at least about 160° C. (about 320° F.) in all four Zones A, B, C, and D—may be held at or above the dry heat sterilizing temperature for a time period of between about 100 seconds and about 600 seconds, and may return to a desirable cooled temperature within a time period of between about 30 seconds and about 600 seconds, and, in some embodiments, the time period is about 100 seconds.

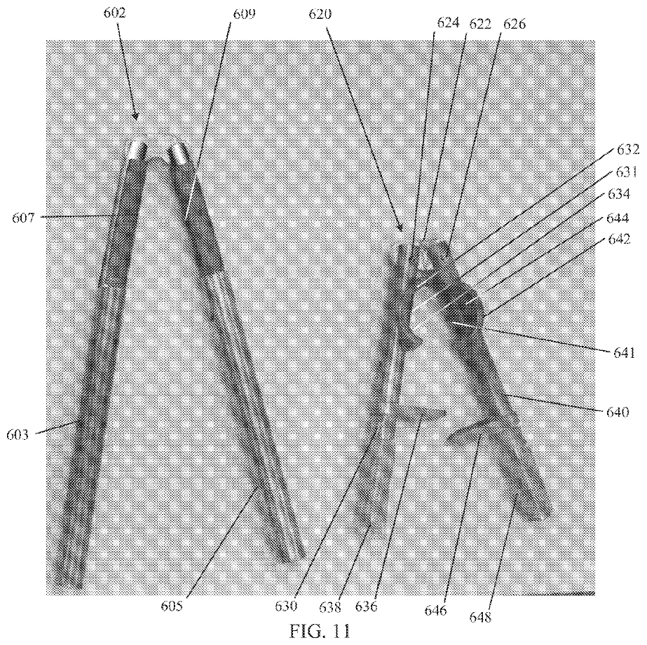

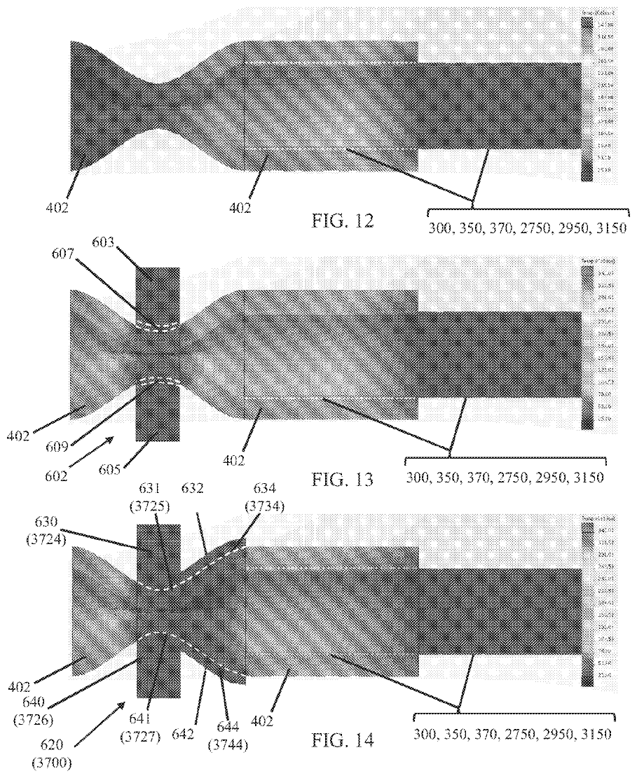

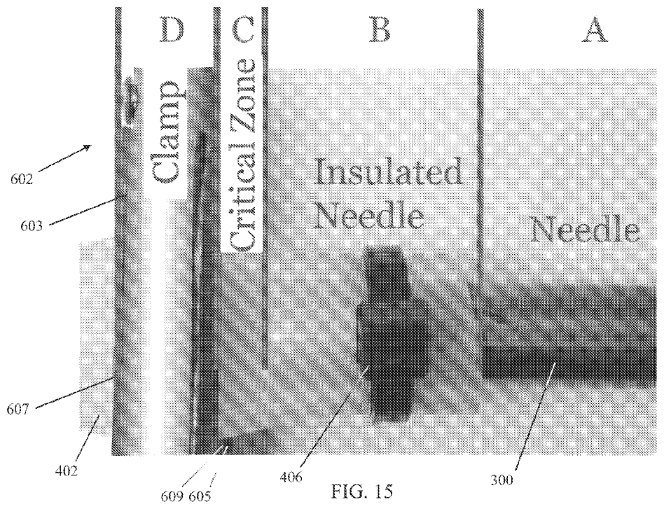

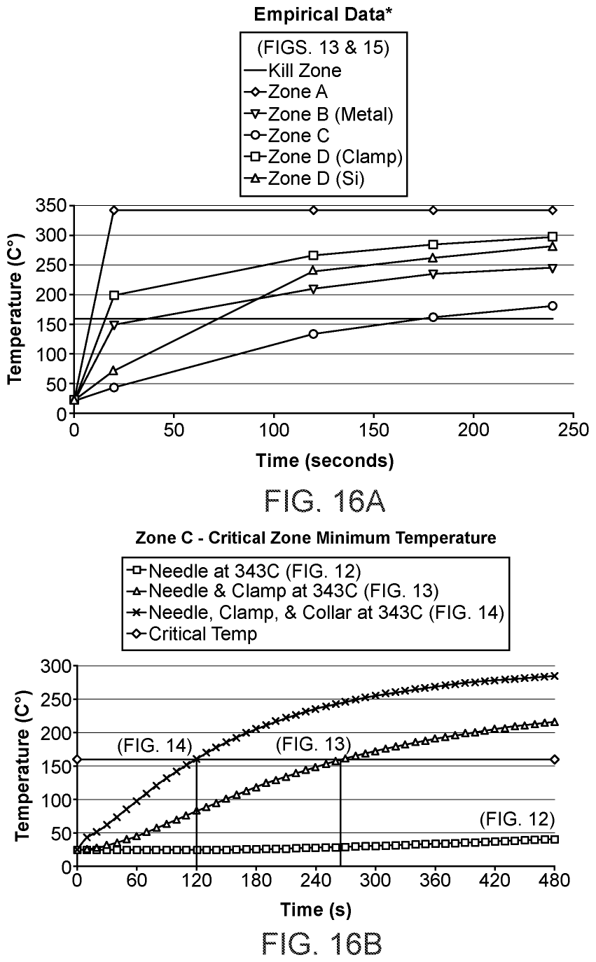

FIG. 16B is a plot of temperature over time of the insert-only configuration of FIG. 12, the insert-plus-clamp configuration of FIG. 13, and the insert-plus-collared-clamp configuration of FIG. 14. Thermal modeling was used to show how temperature at the critical zone (i.e., Zone C) could be improved using collars. In each model, the configurations were modeled at a temperature of 343° C. (649.4° F.). The insert-only configuration of FIG. 12 was unable to achieve the critical temperature of at least 160° C. (320° F.) in Zone C after 8 minutes of heating the insert at 343° C. (649.4° F.). However, the insert-plus-clamp configuration of FIG. 13 was able to achieve the critical temperature of at least 160° C. (320° F.) in Zone C after about 4.4 minutes of heating the insert-plus-clamp at 343° C. (649.4° F.). Moreover, the insert-plus-collared-clamp configuration of FIG. 14 was able to achieve the critical temperature of at least 160° C. (320° F.) in Zone C after about 2.0 minutes of heating the insert-plus-collared-clamp at 343° C. (649.4° F.).

One end of the sterile connection 499 may connect a media bag 700 via the conduit 402, and another end of the sterile connection 499 may connect a piece of equipment (not shown) via the conduit 404.

The conduit 402, 404 may be provided empty, open, filled or unfilled, and/or plugged or unplugged. The conduit 402, 404 may be filled with a liquid. When the conduit 402, 404 is filled with the liquid, a seal may be formed in at least one portion of the conduit 402, 404 and/or on an end 408 thereof (see, FIG. 18). The conduit 402, 404 may be provided plugged or unplugged at one or both ends.

The conduit 402, 404 may include any suitable elastomeric material including silicone having a shore hardness of between about 50 to about 65. The elastomeric material may be pump type silicone having a shore hardness of about 65. The elastomeric material may be reinforced silicone tubing having a shore hardness of between about 65 and about 80. The shore hardness may be any value or subrange within the recited ranges, including endpoints.

The conduit 402, 404 may be cut with a blade. The blade may be a single-use blade. The single-use blade may include a single-use blade material such as SAE 316L grade stainless steel.

Insert or Needle

The insert needle 300 may be used with one or more systems and/or components described herein, such as the first exemplary system, the second exemplary system, the third exemplary system, the process and control methods, the computer-implemented control methods, and/or the like. The insert 300 may be formed of any suitable conductive material including carbon steel or stainless steel. Carbon steel may be selected because it has a property of heating up more quickly than materials such as stainless steel. The insert 300 may be configured to be heated by induction. The insert 300 may be configured to join together two sections of conduit 402, 404.

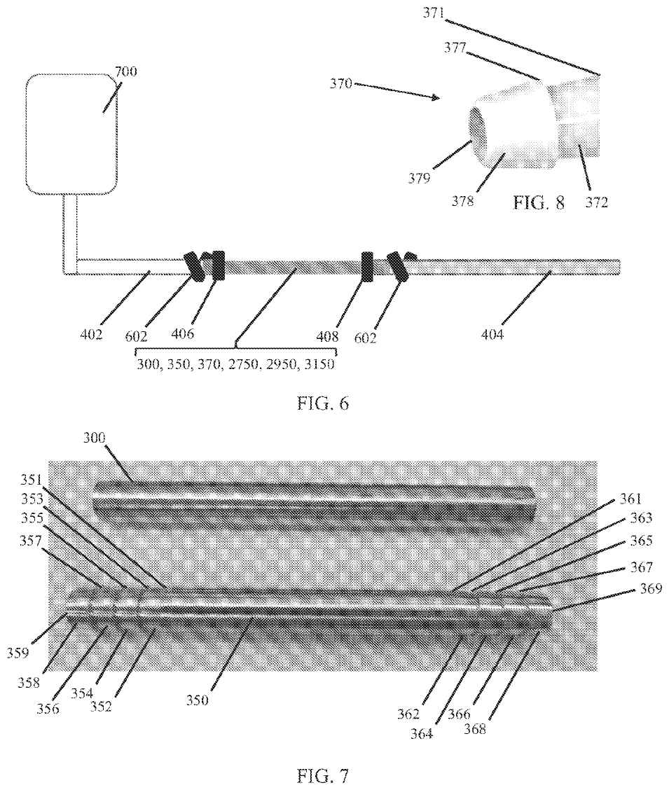

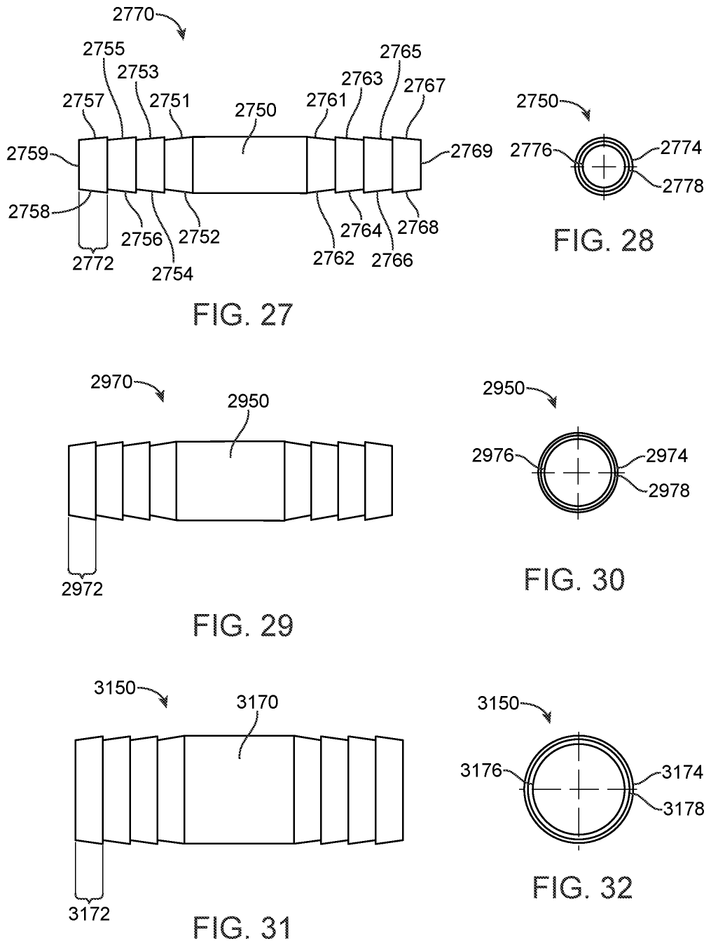

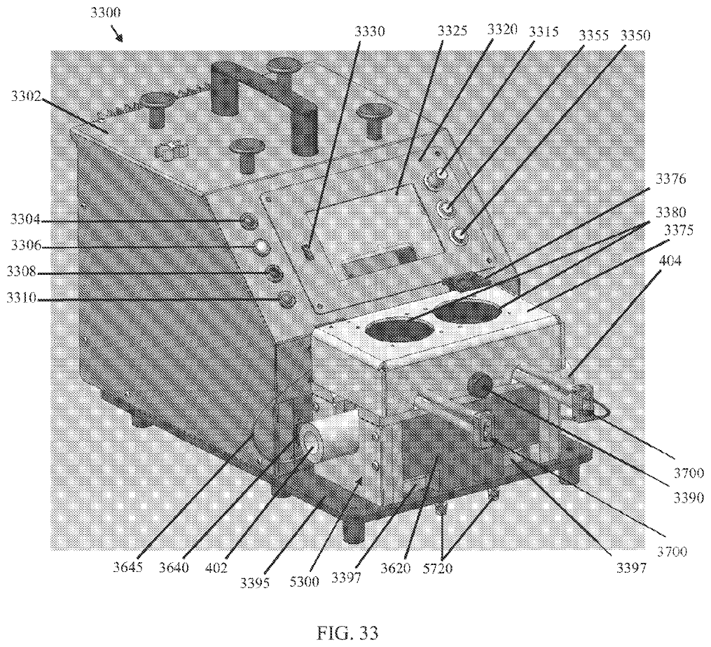

Throughout the present disclosure, reference to an insert or needle may refer to the insert 300 (FIGS. 1, 3, 5, 6, 7, 9, 10, 12-15, 17-19, 21, and 22), a triple barbed insert 350 (FIG. 7), a single barbed insert 370 (FIG. 8), insert 2750 (FIGS. 27 and 28), insert 2950 (FIGS. 29 and 30) and insert 3150 (FIGS. 31 and 32). The insert 300 may have a circular cross section having an outer diameter of between about 0.25 inches (about 0.635 centimeter) to about 1.0 inches (about 2.54 centimeters).

The triple barbed insert 350 may include a generally cylindrical body terminating in a first end 359 and a second end 369. At the first end 359, starting with descriptions near a center of the body and working outward, at an inflection point 351, the insert 350 may include a first inwardly tapered section 352, then a first outwardly extending ridge 353, a second inwardly tapered section 354, then a second outwardly extending ridge 355, a third inwardly tapered section 356, a third outwardly extending ridge 357, and a fourth inwardly tapered section 358 terminating at the first end 359 of the insert 350. The second end 369 may include structures similar to the first end 359, i.e., at the second end 369, starting with descriptions near a center of the body and working outward, at an inflection point 361, the insert 360 may include a first inwardly tapered section 362, then a first outwardly extending ridge 363, a second inwardly tapered section 364, then a second outwardly extending ridge 365, a third inwardly tapered section 366, a third outwardly extending ridge 367, and a fourth inwardly tapered section 368 terminating at the second end 369 of the insert 350. The above-referenced structures form three protruding ridges, and three inward recesses, which provide additional surface area for engagement with the conduits 402, 404.

The single barbed insert 370 includes only one ridge, i.e., starting with descriptions near a center of the body and working outward, at an inflection point 371, the insert 370 may include a first inwardly tapered section 372, then a first outwardly extending ridge 377, a second inwardly tapered section 378 terminating at a first end 379 of the insert 370. A second end of the insert 370 (not shown) may be identical to the first end 370 of the insert 370 but oriented in the opposite direction. The above-referenced structures form one protruding ridge, and one inward recess, which provide additional surface area for engagement with the conduits 402, 404. The inserts 300, 350 and 370 may be varied to include two ridges and two inward recesses, or more than three ridges and more than three inward recesses.

Various tests were performed to prove the efficacy of the above-referenced systems and components in achieving sterilization. For example, a sterility test was conducted using 0.25 inches (0.635 centimeter), 0.5 inches (1.27 centimeters), and 1.0 inches (2.54 centimeters) conduit, examples of which are shown in FIGS. 18 and 19. As shown in FIG. 17, two media bags 700, 700 were connected via two pieces of conduit 402, 404 and the needle 300, which were connected via the process described above to form a sterile connection 499. Also, a tertiary conduit 406 was capped at an end 408 thereof. The cap at the end 408 of the conduit 406, and the connection 499 were made in a non-sterile environment. The media bags 700 contained tryptic soy broth (TSB), which was transferred from one bag 700 to another bag 700 via the connection 499 without a leak. A bacterial agent (e.g., bacillus atrophaeus) was spliced into three locations of the test system of FIG. 17 in amounts of about 10 μL at each location, for a total volume of about 30 μL. If the bacteria was not killed by the dry heat sterilization, the test system would show growth in the TSB media while in the sterile hold. A sterile hold was maintained in a room kept at a temperature of about 37° C. (about 98.6° F.) for about 3 days. A sample of about 50 mL (about 3.051 cubic inches) of the TSB was submitted for detection and identification. Similarly, as shown in FIG. 18, a media bag 700 was connected via conduit 404 to conduit 402 using the insert 300 using a relatively large size of conduit. All samples using 0.25 inches (0.635 centimeter), 0.5 inches (1.27 centimeters), and 1.0 inches (2.54 centimeters) conduit maintained sterility for the entire duration of the tests.

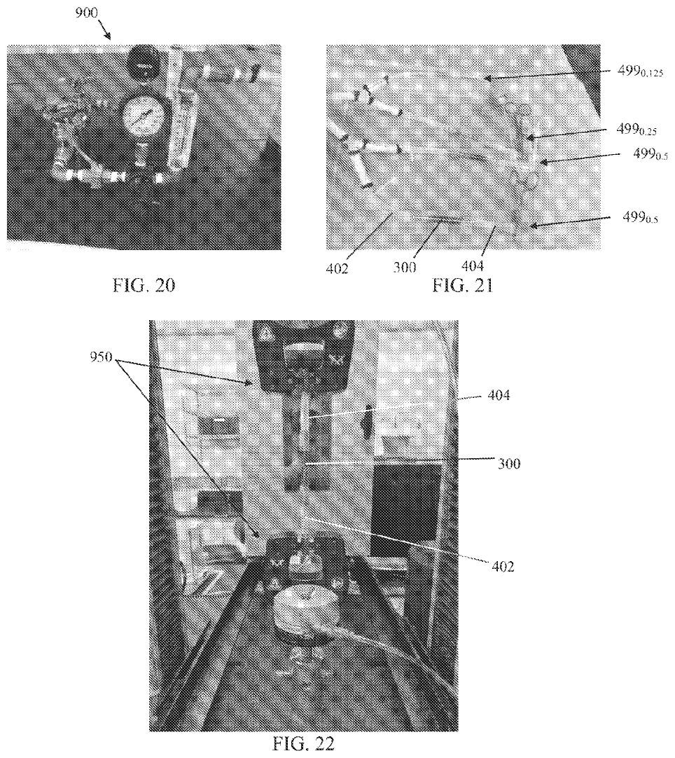

As shown in FIGS. 20 and 21, air leak tests were performed for connections 499 formed with 0.125 inches (0.3175 centimeter) (as shown), 0.25 inches (0.635 centimeter) (as shown), 0.5 inches (1.27 centimeters) (as shown) and 1.0 inches (2.54 centimeters) (not shown) conduits. Each of the connections 499 was connected to an air supply (FIG. 20), pressurized, and submerged in water for about 6 hours. A pressure of about 15 psi (about 1.034e+05 newtons/square meter) was provided to a connection 499_{0.125} using 0.125 inches (0.3175 centimeter) conduit. A pressure of about 15 psi (about 1.034e+05 newtons/square meter) was provided to a connection 499_{0.25} using 0.25 inches (0.635 centimeter) conduit. A pressure of about 45 psi (about 3.103e+05 newtons/square meter) was provided to a connection 499_{0.50} using 0.50 inches (1.27 centimeters) conduit. A pressure of about 45 psi (about 3.103e+05 newtons/square meter) was provided to a connection 499_{1.0} using 1.0 inches (2.54 centimeters) conduit. The connection 499_{0.125} using the 0.125 inches (0.3175 centimeter) conduit and the connection 499_{0.25} using the 0.25 inches (0.635 centimeter) conduit passed at about 15 psi (about 1.034e+05 newtons/square meter). The connection 499_{0.50} using the 0.50 inches (1.27 centimeters) conduit and the connection 499_{1.0} using the 1.0 inches (2.54 centimeters) conduit did not pass at greater than about 25 psi (about 1.724e+05 newtons/square meter). The connection 499_{0.50} using the 0.50 inches (1.27 centimeters) conduit and the connection 499_{1.0} using the 1.0 inches (2.54 centimeters) conduit in which the insert 300 is replaced with either of the barbed inserts 350 and 370 passed at 45 psi (3.103e+05 newtons/square meter). The silicone tubing was secured to the barbs of the barbed inserts 350 and 370 using cable ties.

As shown in FIG. 22, a tensile strength test was performed using an Instron machine 950. Penumatic grips of the Instron machine 950 were set to about 20 psi (about 1.379e+05 newtons/square meter). Connections 499 for each of the four conduit sizes of about 0.125 inches (about 0.3175 centimeter), 0.25 inches (0.635 centimeter), 0.5 inches (1.27 centimeters), and 1.0 inches (2.54 centimeters), were tested using the Instron machine 950, which moved apart at a rate of about 500 mm (about 19.69 inches)/min until failure. A test was deemed successful for the 0.125 inches (0.3175 centimeter) and 0.25 inches (0.635 centimeter) conduits if the connection 499 could withstand greater than about 25 N (about pound force) of force. A test was deemed successful for the 0.50 inches (1.27 centimeters) and 1.0 inches (2.54 centimeters) conduits if the connection 499 could withstand greater than about 50 N (about 11.24 pound force) of force. All four samples passed the tensile strength test.

Process and Control

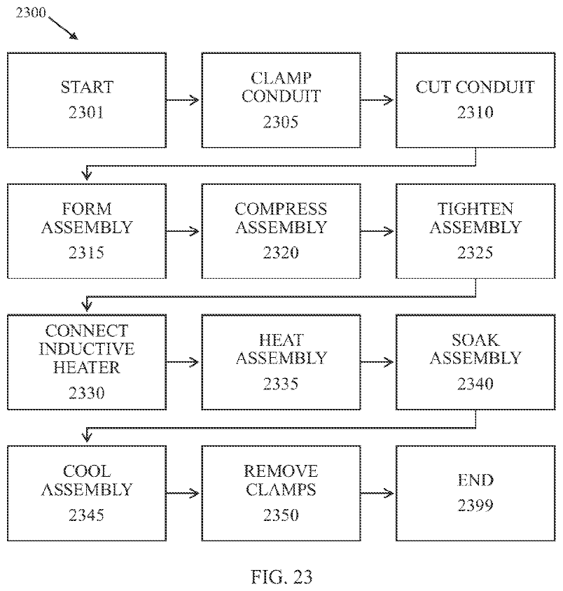

With reference to FIGS. 1, 4, 5, 6, 9, 10, 12-15, 17-19, 21, 22, and 23, an exemplary process 2300 may include multiple steps not included below, duplication of one or more of the steps noted below, and/or elimination of one or more of the steps noted below. The exemplary process 2300 may be used with one or more systems, components, and/or methods described herein, such as the first exemplary system, the second exemplary system, the third exemplary system, the insert or needle, the computer-implemented control methods, and/or the like. The process 2300 may start (2301); each of a first section of conduit 402 and a second section of conduit 404 may be clamped with a clamp 602, 620 (2305); an end of each of the first section of conduit 402 and the second section of conduit 404 may be cut so that about 1.0 inches (about 2.54 centimeters) of conduit extends beyond the clamp 602, 620 (2310); an assembly may be formed, i.e., each end of an insert 300, 350, 370 may be inserted into respective cut ends of the of each of the first section of conduit 402 and the second section of conduit 404 up to a point proximate the clamp 602, 620 so as to form an overlapped region about 1.0 inches (about 2.54 centimeters) in length (2315); a compression ring 406, 408, such as a zip tie, may be placed in an open state over a point proximate the end of each of the first section of conduit 402 and the second section of conduit 404, which itself overlaps the insert 300, 350, 370 (2320); the compression rings 406, 408 may be manipulated into a closed state and tightened in order to seal the conduits 402, 404 to the insert 300, 350, 370 (2325); an inductive heating device 102 may be connected to the insert 300, 350, 370 (2330); the inductive heating device 102 may heat the insert 300, 350, 370 and maintain a predetermined temperature for a duration of time in order to form a sterile connection 499 of an interior of the conduits 402, 404 and the insert 300, 350, 370 (2335); a soaking process (2340); a cooling process (2345); removing the clamps 602, 620 from the sterile connection 499 (2350); and the process may end (2399).

The connecting step 2330 of connecting the inductive heating device 102 to the insert 300, 350, 370 may include connecting the inductive heating device 102 to the clamp 602, 620. The inductive heating device 102 may be indirectly connected to the clamp 602, 620 via the cradle 200.

During the inductive heating step 2335, the conduit 402, 404 may be configured to achieve a dry heat sterilizing temperature of at least about 160° C. (about 320° F.). During the inductive heating step 2335, the conduit 402, 404 and the insert 300, 350, 370 may be configured to achieve a dry heat sterilizing temperature of at least about 160° C. (about 320° F.). During the inductive heating step 2335, the insert 300, 350, 370 may be heated to a temperature of between about 160° C. (about 320° F.) and about 343° C. (about 649.4° F.).

The process 2300 may result in the first conduit 402 and the second conduit 404 being bonded to the insert 300, 350, 370. The process 2300 may result in an assembly or connection 499 including the first conduit 402 and the second conduit 404 being bonded to the insert 300, 350, 370 while maintaining a sterile environment inside the assembly 499.

The process 2300 may include cutting of the conduit 402, 404 (2310). The conduit 402, 404 may be cut with the single-use blade material. Cutting may be performed manually or with an automated external cutting mechanism. Cutting the conduit 402, 404 may have a duration of about 30 seconds.

The process 2300 may include an assembling process including the joining of the conduit 402, 404 and the insert 300, 350, 370 (2315). For example, a first end of an insert 300, 350, 370 may be inserted into a first end of a first conduit 402, and a second end of the insert 300, 350, 370 may be inserted into a first end of a second conduit 404.

The process 2300 may include a heating process (2335), which may also be referred to as a ramping up period. The heating process 2335 may include applying heat to at least the insert 300, 350, 370 with an induction heating device 102. The heating 2335 may be ramped up over a duration of about 30 seconds (i.e., a ramp time). In some exemplary embodiments, an insert 300, 350, 370 may have an outside diameter of greater than about inches (about 1.27 centimeters) and be configured to withstand inductive heating up to a temperature of at least about 343° C. (about 649.4° F.). In some exemplary embodiments, the insert 300, 350, 370 may have an outside diameter of about 0.125 inches (about 0.3175 centimeter) and be configured to withstand inductive heating up to a temperature of at least about 343° C. (about 649.4° F.). The induction heating device 102 may be powered with a power source having an output of about 120 volts AC. The heating process 2335 may be performed for a duration of time between about 20 seconds and about 300 seconds. The duration may be any value or subrange within the recited ranges, including endpoints.

The process 2300 may include a soaking process 2340. Soaking as used herein in this context refers to a time period (e.g., at least about 100 seconds) during which a specified temperature is held. In some exemplary embodiments, the soaking process 2340 may have a duration of about 120 seconds. In some exemplary embodiments, an insert 300, 350, 370 may have an outside diameter of greater than about 0.5 inches (about 1.27 centimeters) and a duration of the soaking process 2340 may be between about 100 seconds and about 600 seconds.

The process 2300 may include a cooling process 2345. The cooling process 2345 may have a duration of between about 30 seconds and about 600 seconds. The cooling process 2345 may be performed with forced air cooling, e.g., via a cooling device 580. The cooling process 2345 may achieve a reduction in a temperature of the insert to about 50° C. (about 122° F.) or less. A temperature measurement system may be provided to monitor the temperature of the insert 300, 350, 370. The temperature measurement system may include a signal configured to send a signal when the temperature drops to about 50° C. (about 122° F.) or less.

A total duration of the process 2300 including the cutting process 2310, the assembling process 2315, the heating process 2335, the soaking process 2340, and the cooling process 2345, including optional pauses between steps may be between about 3.0 minutes and about 15.0 minutes. In some embodiments, the duration of the process 2300 is between about 5.0 minutes and about 7.0 minutes. In particular embodiments, the duration of the process 2300 is about 300 seconds (5.0 minutes).

The process 2300 may by manually performed by a technician without automation. The process 2300 may be semi-automated or fully automated with a system configured to perform one or more of the cutting process 2310, the assembling process 2315, the heating process 2335, the soaking process 2340, and the cooling process 2345. Any one or more of the steps may be excluded from the process 2300. Additional steps may be added, in addition to or in lieu of the recited steps.

The cutting process 2310 may include a cutting mechanism configured to cut the conduit 402, 404. The cutting mechanism may be manually operated by a technician without automation. The cutting process may be semi-automated or fully automated with the cutting mechanism configured to cut the conduit. The cutting mechanism may include disposable blades. The disposable blades may be heated prior to cutting the conduit 402, 404. The cutting mechanism may be configured to cut the conduit 402, 404 in a manner that minimizes particle shedding.

The assembling process 2315 of the process 2300 may include a loading process 2317. The loading process 2317 may be manually performed by a technician without automation. The loading process 2317 may include a loading mechanism configured to place a first conduit 402 onto a first end of the insert 300, 350, 370 and a second conduit 404 onto a second end of the insert 300, 350, 370.

The system 100, 500 may include a safety mechanism (e.g., 575) configured to prevent contact with sharp, hot and moving parts. The safety mechanism 575 may be a safety shield provided over an area where the heating process is performed. The safety shield 575 may be configured for operation in a closed, protective position and in an open, non-protective position. The safety shield 575 may be configured to activate the cooling process when placed into the open, non-protective position. The safety mechanism 575 may be configured to cover parts of the system 100, 500 having a temperature exceeding about 50° C. (about 122° F.) during processing.

The process 2300 may be configured to maintain sterility of conduits 402, 404, including inner surfaces of the conduits 402, 404 and any process fluid that may be provided therein. Specifically, the system 100, 500 may be configured to ensure sufficient temperatures are achieved and held for sufficient durations of time to achieve sterilization of the insert 300, 350, 370 and the conduit 402, 404. A risk of direct contact of the process fluid with the heating area is reduced by the configuration of the components of the system 100, 500. Also, the risk is reduced by an operator exercising appropriate caution during processing.

A touch screen (e.g., 105, 520) may be provided for controlling one or more components of the system 100, 500. In some exemplary embodiments, the touch screen may be a standard universal personal telecommunications (UPT) liquid crystal display (LCD) screen with an overlay for a keypad. The touch screen may include status indicators for a given process, e.g., ramp, soak, cool, and the like.

A temperature controller may be provided. The temperature controller may be configured to control a temperature of inductive heat generated at a given component of the system 100, 500, e.g., the insert 300, 350, 370. The temperature controller may be configured to control the temperature in a range from an ambient condition (e.g., about 20° C. (about 68° F.)) to about 500° C. (about 932° F.). The temperature controller may be configured to control the temperature in increments, e.g., +/−5° C. (41° F.) increments. The temperature may be any value or subrange within the recited ranges, including endpoints.

The temperature controller may include an infrared system may be provided to monitor the temperature. The infrared system may be configured to detect temperatures in the range from the ambient condition (e.g., about 20° C. (about 68° F.)) to about 500° C. (about 932° F.). In other embodiments, the infrared system may be configured to detect temperatures in a range from about 0° C. (about 32° F.) to about 500° C. (about 932° F.). The temperature may be any value or subrange within the recited ranges, including endpoints.

The temperature controller may include display of a set point temperature (i.e., a target temperature) and a current temperature via the UPT LCD. The set point and the current temperatures may be displayed simultaneously. The temperature controller may include a calibration procedure for calibrating the temperature control system. For example, for each new component, such as a new insert, a temperature calibration may be desirable, and may be required to ensure maximum accuracy.

A time display may be provided configured to display a time remaining for a current process, e.g., a timer for a soak cycle or a cool cycle. In some exemplary embodiments, a countdown timer may be displayed for the soak cycle, and a count-up timer may be displayed for the cool cycle.

A time control may be provided for each process (e.g., ramp, soak, cool, and the like). For example, a plurality of time control programs may be provided based on a size and/or a type of an insert, e.g., 0.125 inches (0.3175 centimeter), 0.25 inches (0.635 centimeter), 0.5 inches (1.27 centimeters), and/or an insert for processing of plasmids (e.g., pRG4, pRG5), and the like. The ramp time may not be a specific time. The ramp time may be dependent on a time to achieve a given temperature based on a power and a size of a part. The soak time may be set to run once a part achieves a given temperature. The cool time may be set using a timer. The names of the stages of the process (e.g., ramp, soak, cool, and the like) may be changed by a user or locked into firmware. Parameters of the process may be user programmed, so a given process may be modified as needed. The system may be configured to allow a user to control, e.g., power output, and the time of each stage of the process, including optional pauses between stages. Programs may be stored. Programs may be customized for a particular size of conduit and/or to achieve a particular temperature profile within the connection.

A notification system may be provided. The notification system may include indicators including visual signals and audible signals. The visual and audible signals may indicate a status of the system 100, 500.

An emergency stop mechanism (e.g., 120, 550) may be provided. The emergency stop mechanism may be configured to stop all physical movement of the system 100, 500 immediately. The emergency stop mechanism may be located in an easily accessible area proximate the system 100, 500. The emergency stop mechanism may have no moving parts, e.g., a “Heat Off” button may be provided or displayed on a control panel, and the button may be configured to stop the heat cycle and open the safety shield.

A lockout mechanism may be provided. The lockout mechanism may be accompanied with a tagout procedure including placement of a tagout tag. The lockout mechanism may be provided as part of a circuit breaker of a power supply.

The system 100, 500 may include an operation system. The operation system may include an automatic mode and a manual mode. The operation system may be configured to give a user access to one or more functions of the system 100, 500 and/or locked out of one or more functions of the system 100, 500. The operation subsystem may include built-in operating programs for commonly used tubing types and sizes. For example, the operation system may include five automatic heat/cool modes for each of the five insert types noted above, i.e., 0.125 inches (0.3175 centimeter), 0.25 inches (0.635 centimeter), 0.5 inches (1.27 centimeters), pRG4 and pRG5. The operation system may be configured with or without user rights for setting recipes.

The operation system may be configured to prompt the user during operation.

The operation system may include options to add new operating programs. For example, recipes for each of the five automatic heat/cool modes for each of the five insert types noted above, i.e., 0.125 inches (0.3175 centimeter), 0.25 inches (0.635 centimeter), inches (1.27 centimeters), pRG4 and pRG5, may be adjusted. The operation system may include an emergency stop program, which immediately cuts power to the entire system 100, 500. The emergency stop program may include a “Heat Off” button provided or displayed on a control panel, and the button may be configured to stop the heat cycle and open the safety shield.

Critical alarms may be provided to take action. The critical alarms may include an emergency stop, a control power fault, a motor fault and a temperature fault. In some exemplary embodiments, when a critical alarm is triggered, an interlock or plurality of interlocks may be activated to shut down and/or power off equipment. In addition or in lieu of the interlock(s), a user may be prompted to perform an operator procedural response, which may include shut down and/or power off. The system 100, 500 may be configured to notify the operator of one or more conditions of the system 100, 500. The operator may be required to acknowledge the alarm before the alarm can be reset and the system 100, 500 restarted. Once the alarm is reset, the operator may restart the system 100, 500. The power off operation may be limited to an induction power supply.

Programs and data may be stored in the system 100, 500 and accessible for download via USB connection. The data may include temperature data, duration of bonding steps, dates, and the like. Data packets may be transmitted via a serial interface for data logging. The data may be transmitted in accordance with Recommended Standard 232 (RS-232). An external device 800 may be connected to the system 100, 500 for collecting and storing data (FIG. 17). The external device 800 may include logging software. The system 100, 500 may be compliant with Title 21 of the Code of Federal Regulations, the rules of the Food and Drug Administration.

The system 100, 500 may be provided in an enclosure. In an exemplary embodiment, the enclosure may have a vertical clearance of about 12 to 24 inches (about to 60.96 centimeters), e.g., about 15 inches (about 38.1 centimeters), a horizontal clearance of about 8 to 24 inches (about 20.32 to 60.96 centimeters), e.g., about 12 inches (about 30.48 centimeters), and a depth clearance of about 12 to 24 inches (about 30.48 to centimeters), e.g., about 13 inches (about 33.02 centimeters). The system 100, 500 may have a total weight of less than about 50 pounds (about 22.68 kilograms), e.g., about 44 pounds (about 19.96 kilograms). The system 100, 500 may be configured for operation within an ambient temperature range of from about 0° C. (about 32° F.) to about 40° C. (about 104° F.). One or more parts of the system 100, 500 may be sterilized with a mixture of 70% pure isopropyl alcohol and 30% water (70/30 IPA). The system 100, 500 may be configured for a wipe down cleaning, but may not necessarily be washed down or immersed. The system 100, 500 may be configured without sharp edges to avoid the risk of a glove of a user catching on a sharp edge and being torn during operation. The system 100, 500 may include a mechanism for storing a power cord. The system 100, 500 may include a label or tag bearing identifying information including model and revision numbers, a serial number, line voltage and frequency and supplier contact information.

An electrical system may be provided. The system 100, 500 may operate on a power source having 15 Amp, 120 volts AC at 60 Hz, single phase. The system 100, 500 may include an uninterruptible power supply (UPS). The system 100, 500 may include a waterproof and UL rated power cord having a length of about 6 feet (about 1.829 meters). The system 100, 500 may be configured for operation on a regular basis for about 8 hours per day and up to 7 days per week.

One or more components of the system 100, 500 may be coated with a thermally resistant magnesium oxychloride (MOC) coating.

Computer-Implemented Control

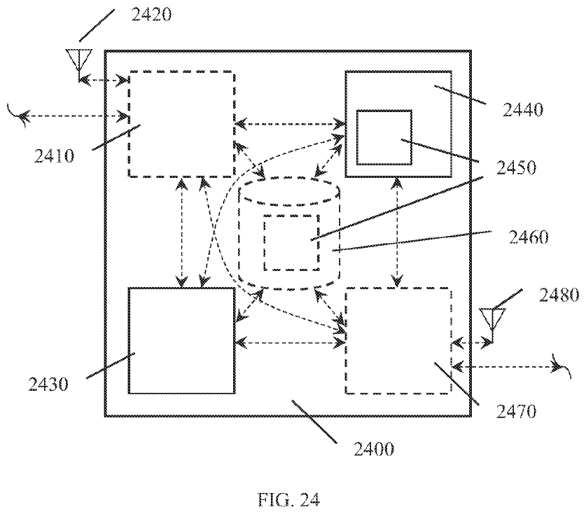

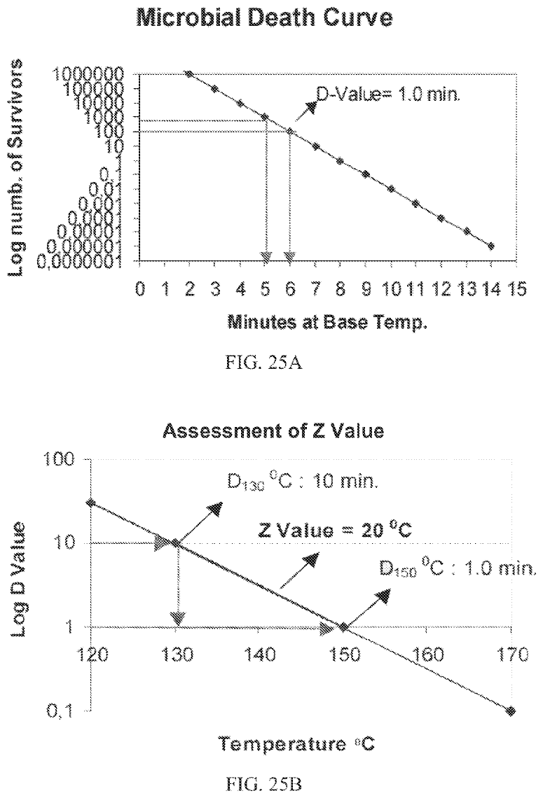

FIG. 24 is a schematic diagram of a computer device or system including at least one processor and a memory storing at least one program for execution by the at least one processor according to an exemplary embodiment. Specifically, FIG. 24 depicts a computer device or system 2400 comprising at least one processor 2430 and a memory 2440 storing at least one program 2450 for execution by the at least one processor 2430. The computer device or system 2400 may be used with one or more systems and/or components described herein, such as the first exemplary system, the second exemplary system, the third exemplary system, the insert or needle, the process and control methods, the computer-implemented control methods, and/or the like. In some embodiments, the device or computer system 2400 can further include a non-transitory computer-readable storage medium 2460 storing the at least one program 2450 for execution by the at least one processor 2430 of the device or computer system 2400. In some embodiments, the device or computer system 2400 can further include at least one input device 2410, which can be configured to send or receive information to or from any one of: an external device (not shown), the at least one processor 2430, the memory 2440, the non-transitory computer-readable storage medium 2460, and at least one output device 2470. The at least one input device 2410 can be configured to wirelessly send or receive information to or from the external device via a means for wireless communication, such as an antenna 2420, a transceiver (not shown) or the like. In some embodiments, the device or computer system 2400 can further include at least one output device 2470, which can be configured to send or receive information to or from any one from the group consisting of: an external device (not shown), the at least one input device 2410, the at least one processor 2430, the memory 2440, and the non-transitory computer-readable storage medium 2460. The at least one output device 2470 can be configured to wirelessly send or receive information to or from the external device via a means for wireless communication, such as an antenna 2480, a transceiver (not shown) or the like.

The at least one program 2450 may include one or more instructions including one or more steps of the exemplary process 2300. The instructions of the at least one program 2450 may include multiple steps not included in the process 2300 above, duplication of one or more of the steps of the process 2300 above, and/or elimination of one or more of the steps of the process 2300 above. In particular, control of the heating step 2335, the soaking step 2340, the cooling step 2345, and timers associated with each of these steps may be performed by the at least one program 2450. The input device 2410 may be a sensor, such a temperature sensor, a timer, the external device 800, or any other suitable component of the system 100, 500. The output device may be one or more of the displays of the system 100, 500 described above the external device 800, or any other suitable component of the system 100, 500. The controller 110 may be part of the computer device or system 2400 or separate therefrom.

Each of the above identified modules or programs corresponds to a set of instructions for performing a function described above. These modules and programs (i.e., sets of instructions) need not be implemented as separate software programs, procedures or modules, and thus various subsets of these modules may be combined or otherwise re-arranged in various embodiments. In some embodiments, memory may store a subset of the modules and data structures identified above. Furthermore, memory may store additional modules and data structures not described above.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described herein can include electrical circuit(s) that can include components and circuitry elements of suitable value in order to implement the embodiments of the subject innovation(s). Furthermore, it can be appreciated that many of the various components can be implemented on at least one integrated circuit (IC) chip. For example, in one embodiment, a set of components can be implemented in a single IC chip. In other embodiments, at least one of respective components are fabricated or implemented on separate IC chips.

What has been described above includes examples of the embodiments of the present invention. It is, of course, not possible to describe every conceivable combination of components or methodologies for purposes of describing the claimed subject matter, but it is to be appreciated that many further combinations and permutations of the subject innovation are possible. Accordingly, the claimed subject matter is intended to embrace all such alterations, modifications, and variations that fall within the spirit and scope of the appended claims. Moreover, the above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In particular and in regard to the various functions performed by the above described components, devices, circuits, systems and the like, the terms used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the innovation includes a system as well as a computer-readable storage medium having computer-executable instructions for performing the acts and/or events of the various methods of the claimed subject matter.

The aforementioned systems/circuits/modules have been described with respect to interaction between several components/blocks. It can be appreciated that such systems/circuits and components/blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that at least one component may be combined into a single component providing aggregate functionality or divided into several separate sub-components, and any at least one middle layer, such as a management layer, may be provided to communicatively couple to such sub-components in order to provide integrated functionality. Any components described herein may also interact with at least one other component not specifically described herein but known by those of skill in the art.

As used in this application, the terms “component,” “module,” “system,” or the like are generally intended to refer to a computer-related entity, either hardware (e.g., a circuit), a combination of hardware and software, software, or an entity related to an operational machine with at least one specific functionality. For example, a component may be, but is not limited to being, a process running on a processor (e.g., digital signal processor), a processor, an object, an executable, a thread of execution, a program, and/or a computer. By way of illustration, both an application running on a controller and the controller can be a component. At least one component may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

Computing devices typically include a variety of media, which can include computer-readable storage media and/or communications media, in which these two terms are used herein differently from one another as follows. Computer-readable storage media can be any available storage media that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by at least one local or remote computing device, e.g., via access requests, queries or other data retrieval protocols, for a variety of operations with respect to the information stored by the medium.

On the other hand, communications media typically embody computer-readable instructions, data structures, program modules or other structured or unstructured data in a data signal that can be transitory such as a modulated data signal, e.g., a carrier wave or other transport mechanism, and includes any information delivery or transport media. The term “modulated data signal” or signals refers to a signal that has at least one of its characteristics set or changed in such a manner as to encode information in at least one signal. By way of example, and not limitation, communication media include wired media, such as a wired network or direct-wired connection, and wireless media such as acoustic, RF, infrared and other wireless media.

In view of the exemplary systems described above, methodologies that may be implemented in accordance with the described subject matter will be better appreciated with reference to the flowcharts of the various figures. For simplicity of explanation, the methodologies are depicted and described as a series of acts. However, acts in accordance with this disclosure can occur in various orders and/or concurrently, and with other acts not presented and described herein. Furthermore, not all illustrated acts may be required to implement the methodologies in accordance with the disclosed subject matter. In addition, those skilled in the art will understand and appreciate that the methodologies could alternatively be represented as a series of interrelated states via a state diagram or events. Additionally, it should be appreciated that the methodologies disclosed in this specification are capable of being stored on an article of manufacture to facilitate transporting and transferring such methodologies to computing devices. The term article of manufacture, as used herein, is intended to encompass a computer program accessible from any computer-readable device or storage media.

Dry Heat Sterilization Calculations

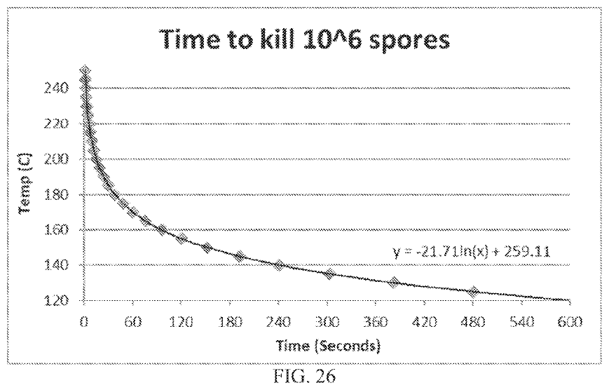

To ensure that a probability of survival of a native microflora in a connection of silicone tubing via insert is no greater than one cell in one million units of a given commodity, i.e., a 10-6 probability of nonsterility, a D-value (FIG. 25A), and a Z-value (FIG. 25B) were calculated and used to generate a time to kill 10⁶ spores curve at various temperatures (FIG. 26). For most purposes, it is considered overkill to achieve excess of 10⁻⁶ probability.

The D-value is the time required to reduce a population of an organism by 90% (one log) at a base temperature. Z-values include a constant death rate log of D-value versus temperature. The Z-value may be assumed to be 20° C. (68° F.). The following formula was used:

Fh: ΔT×Lethality rate: (10\hat{ }((T−Tb)/Z)

Fh is a temperature for dry heat sterilization, ΔT is a cycle time, T is an actual cycle temperature, Tb is a base temperature, and Z is a microbial death rate constant.

The model assumes equivalent minutes at 170° C. (338° F.), 1 log at a given temperature, the organism is b. subtilis, which is sterilized using dry heat sterilization.

At the relatively low temperature of about 120° C. (about 248° F.), a time to kill about 10⁶ spores was about 600 seconds (about 10 minutes); at about 140° C. (about 284° F.), the time to kill about 10⁶ spores was about 240 seconds (about 4 minutes); and at about 200° C. (about 392° F.), the time to kill about 10⁶ spores was about 18 seconds (about 0.3 minutes) (steam).

Additional Exemplary Inserts (Needles)

The inserts 2750, 2950 and 3150 of FIGS. 27-32 are similar in some regards to the insert 350 of FIG. 7, and may be used in conjunction with and/or in place of the insert 350 and/or with one another. Although three barbs are illustrated, less (including none) or more barbs may be provided and are within the scope of the present disclosure. Please note, the last two numbers of each reference number in the descriptions of FIGS. 27-32 below are used to identify structures and features that are relatively similar to those of FIG. 7 having the same last two reference numbers.

FIG. 27 is a side view of a fourth insert 2750 according to an exemplary embodiment. The triple barbed insert 2750 may include a generally cylindrical body terminating in a first end 2759 and a second end 2769. At the first end 2759, starting with descriptions near a center of the body and working outward, at an inflection point 2751, the insert 2750 may include a first inwardly tapered section 2752, then a first outwardly extending ridge 2753, a second inwardly tapered section 2754, then a second outwardly extending ridge 2755, a third inwardly tapered section 2756, a third outwardly extending ridge 2757, and a fourth inwardly tapered section 2758 terminating at the first end 2759 of the insert 2750. The second end 2769 may include structures similar to the first end 2759, i.e., at the second end 2769, starting with descriptions near a center of the body and working outward, at an inflection point 2761, the insert 2760 may include a first inwardly tapered section 2762, then a first outwardly extending ridge 2763, a second inwardly tapered section 2764, then a second outwardly extending ridge 2765, a third inwardly tapered section 2766, a third outwardly extending ridge 2767, and a fourth inwardly tapered section 2768 terminating at the second end 2769 of the insert 2750. The above-referenced structures form three protruding ridges, and three inward recesses, which provide additional surface area for engagement with the conduits 402, 404.

The fourth insert 2750 may have a length 2770 of about 3.00 inches (about 7.62 centimeters), although shorter or longer lengths are within the scope of the present disclosure. Each of the inwardly tapered sections 2752, 2754, 2756, and 2758 may have a length 2772 of about 0.25 inches (about 0.635 centimeter), although shorter or longer lengths are within the scope of the present disclosure.

FIG. 28 is an end view of the fourth insert 2750 according to an exemplary embodiment. The fourth insert 2750 may have an outside diameter 2774 of about 0.50 inches (about 1.27 centimeters), and an inside diameter 2776 of about 0.37 inches (about 0.9398 centimeter) (a thickness of about 0.13 inches (about 0.3302 centimeter)), although smaller or larger diameters and thicknesses are within the scope of the present disclosure. The fourth insert 2750 may have a tapered diameter 2778 at the ridge 2753, the ridge 2755, the ridge 2757, and the end 2759 of about 0.44 inches (about 1.118 centimeters) (a thickness of about 0.07 inches (about 0.1778 centimeter)), although smaller or larger diameters and thicknesses are within the scope of the present disclosure.

FIG. 29 is a side view of a fifth insert 2950 according to an exemplary embodiment (like references are omitted from FIG. 29 but understood as similar to FIG. 27). The triple barbed insert 2950 may include a generally cylindrical body terminating in a first end 2959 and a second end 2969. At the first end 2959, starting with descriptions near a center of the body and working outward, at an inflection point 2951, the insert 2950 may include a first inwardly tapered section 2952, then a first outwardly extending ridge 2953, a second inwardly tapered section 2954, then a second outwardly extending ridge 2955, a third inwardly tapered section 2956, a third outwardly extending ridge 2957, and a fourth inwardly tapered section 2958 terminating at the first end 2959 of the insert 2950. The second end 2969 may include structures similar to the first end 2959, i.e., at the second end 2969, starting with descriptions near a center of the body and working outward, at an inflection point 2961, the insert 2960 may include a first inwardly tapered section 2962, then a first outwardly extending ridge 2963, a second inwardly tapered section 2964, then a second outwardly extending ridge 2965, a third inwardly tapered section 2966, a third outwardly extending ridge 2967, and a fourth inwardly tapered section 2968 terminating at the second end 2969 of the insert 2950. The above-referenced structures form three protruding ridges, and three inward recesses, which provide additional surface area for engagement with the conduits 402, 404.

The fourth insert 2950 may have a length 2970 of about 3.00 inches (about 7.62 centimeters), although shorter or longer lengths are within the scope of the present disclosure. Each of the inwardly tapered sections 2952, 2954, 2956, and 2958 may have a length 2972 of about 0.25 inches (about 0.635 centimeter), although shorter or longer lengths are within the scope of the present disclosure.

FIG. 30 is an end view of the fourth insert 2950 according to an exemplary embodiment. The fourth insert 2950 may have an outside diameter 2974 of about 0.75 inches (about 1.905 centimeters), and an inside diameter 2976 of about 0.62 inches (about 1.575 centimeters) (a thickness of about 0.13 inches (about 0.3302 centimeter)), although smaller or larger diameters and thicknesses are within the scope of the present disclosure. The fourth insert 2950 may have a tapered diameter 2978 at the ridge 2953, the ridge 2955, the ridge 2957, and the end 2959 of about 0.69 inches (about 1.753 centimeters) (a thickness of about 0.07 inches (about 0.1778 centimeter)), although smaller or larger diameters and thicknesses are within the scope of the present disclosure.

FIG. 31 is a side view of a sixth insert 3150 according to an exemplary embodiment (like references are omitted from FIG. 31 but should be understood as similar to FIG. 27). The triple barbed insert 3150 may include a generally cylindrical body terminating in a first end 3159 and a second end 3169. At the first end 3159, starting with descriptions near a center of the body and working outward, at an inflection point 3151, the insert 3150 may include a first inwardly tapered section 3152, then a first outwardly extending ridge 3153, a second inwardly tapered section 3154, then a second outwardly extending ridge 3155, a third inwardly tapered section 3156, a third outwardly extending ridge 3157, and a fourth inwardly tapered section 3158 terminating at the first end 3159 of the insert 3150. The second end 3169 may include structures similar to the first end 3159, i.e., at the second end 3169, starting with descriptions near a center of the body and working outward, at an inflection point 3161, the insert 3160 may include a first inwardly tapered section 3162, then a first outwardly extending ridge 3163, a second inwardly tapered section 3164, then a second outwardly extending ridge 3165, a third inwardly tapered section 3166, a third outwardly extending ridge 3167, and a fourth inwardly tapered section 3168 terminating at the second end 3169 of the insert 3150. The above-referenced structures form three protruding ridges, and three inward recesses, which provide additional surface area for engagement with the conduits 402, 404.

The fourth insert 3150 may have a length 3170 of about 3.00 inches (about 7.62 centimeters), although shorter or longer lengths are within the scope of the present disclosure (for example, about 2.42 inches (about 6.147 centimeters)). Each of the inwardly tapered sections 3152, 3154, 3156, and 3158 may have a length 3172 of about 0.25 inches (about 0.635 centimeter), although shorter or longer lengths are within the scope of the present disclosure.

FIG. 32 is an end view of the fourth insert 3150 according to an exemplary embodiment. The fourth insert 3150 may have an outside diameter 3174 of about 1.00 inches (about 2.54 centimeters), and an inside diameter 3176 of about 0.87 inches (about 2.21 centimeters) (a thickness of about 0.13 inches (about 0.3302 centimeter)), although smaller or larger diameters and thicknesses are within the scope of the present disclosure. The fourth insert 3150 may have a tapered diameter 3178 at the ridge 3153, the ridge 3155, the ridge 3157, and the end 3159 of about 0.94 inches (about 2.388 centimeters) (a thickness of about 0.07 inches (about 0.1778 centimeter)), although smaller or larger diameters and thicknesses are within the scope of the present disclosure.

Third Exemplary System

A third generation system (e.g., FIGS. 33-63) for sterilization by inductive heating is disclosed hereinbelow. Please note, any of the structures and features of the systems, components, processes, and control systems disclosed above may be used in combination with or in lieu of comparable structures and features of the third system disclosed below, and vice versa.

FIG. 33 is a first perspective view of a third system 3300 for making a sterile connection between at least one conduit (e.g., 402, 404) and an insert 300, 350, 370, 2750, 2950, 3150 including an induction heating device 3302, a third cradle 5300, and a pair of third clamps 3700 according to an exemplary embodiment.

A third system 3300 may include a housing of an inductive heating device 3302. The cradle 5300 may be attached to the housing of the inductive heating device 502 or provided separate therefrom and slid into and out of a position proximate the housing of the inductive heating device 3302. Portions of the inductive heating device 3302 may be contained within the housing or provided separately therefrom.

The housing of the inductive heating device 3302 may include four indicators 3304, 3306, 3308, and 3310, which may correspond with stages of the inductive heating process. In some embodiments, the first indicator 3304 may be configured to emit green light to visually indicate a “READY” state, the second indicator 3306 may be configured to emit yellow light to visually indicate a “HEATING” stage, the third indicator 3308 may be configured to emit blue light to visually indicate a “CYCLE COOLING” stage, and the fourth indicator 3310 may be configured to emit red light to visually indicate an “ERROR” state.

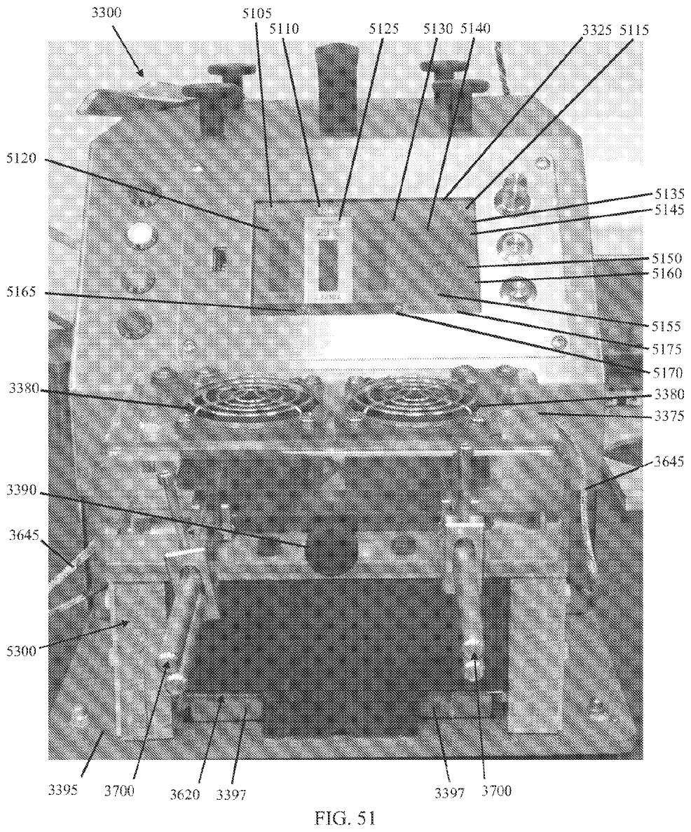

The third system 3300 may include a rotatable selection knob or dial 3315. A touch screen display 3325 may be mounted beneath a protective plate 3320. A USB connection port 3330 may be provided. A stop button 3350 and a start button 3355 may be provided. Additional features of the touch screen display 3325 are described with reference to FIG. 51 below.

A platform 3395 may support the third system 3300. The cradle 5300 may be mounted to the platform 3395. One or more clamp bases 3397 may be mounted to the platform 3395. The clamp bases 3397 may be configured to support an end of each of the third clamps 3700. A protective cover 3375 may be provided to enclose the conduits 402, 404, the insert 300, 350, 370, 2750, 2950, 3150, inductive heating elements (described below), the third clamps 3700, and the cradle 5300. One or more cooling fans 3380 may be connected to an inner surface of the protective cover 3375. A knob 3390 may be provided on the protective cover 3375 to facilitate opening and closing of the protective cover 3375. The protective cover 3375 may be connected to the housing of the inductive heating device 3302 via one or more hinges 3376.

One or more induction blocks 3620 may surround the connection of the conduits 402 and 404 to be sterilized. The induction blocks 3620 facilitate generation of inductive heat in adjacent metallic components including, e.g., the insert 300, 350, 370, 2750, 2950, 3150, the third clamps 3700, and the like. One or more thermocouples 3640 may be connected via wiring 3645 to points within the third system 3300 to detect temperature at desired points, e.g., a temperature inside each of the conduits 402, 404, a temperature of the insert 300, 350, 370, 2750, 2950, 3150, a temperature of the third clamps 3700, and the like. A pair of hex nuts 5720 are visible below the platform 3395 (to be described in greater detail below with reference to FIG. 57).

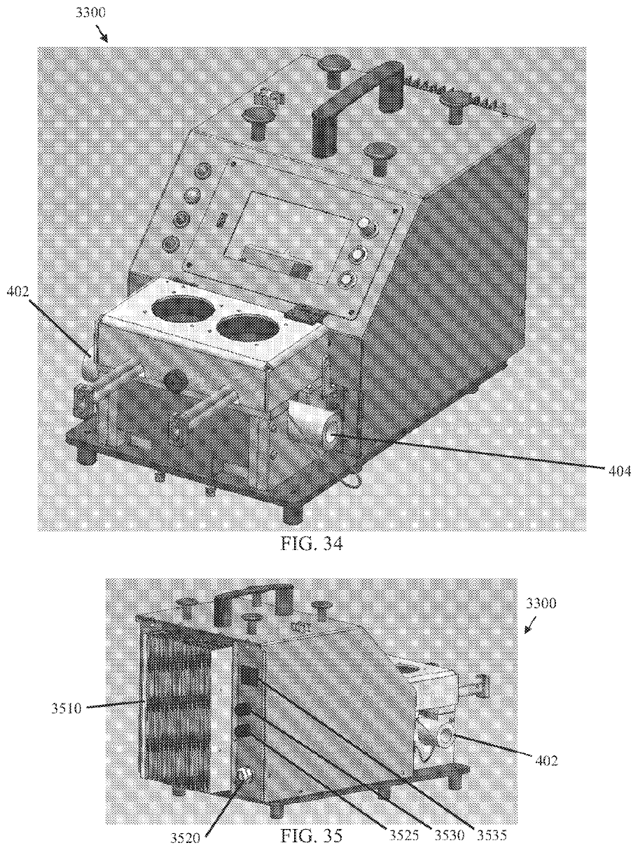

FIG. 34 is a second perspective view of the third system 3300 according to an exemplary embodiment.

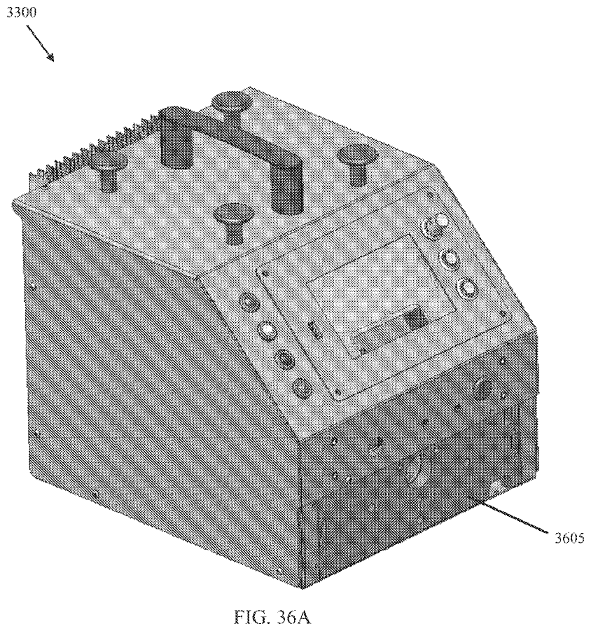

FIG. 35 is a rear perspective view of the third system 3300 according to an exemplary embodiment. The third system 3300 may include one or more of a cooling radiator 3510, a first fuse 3520, a second fuse 3525, a third fuse 3530, and an on/off power toggle switch 3535.

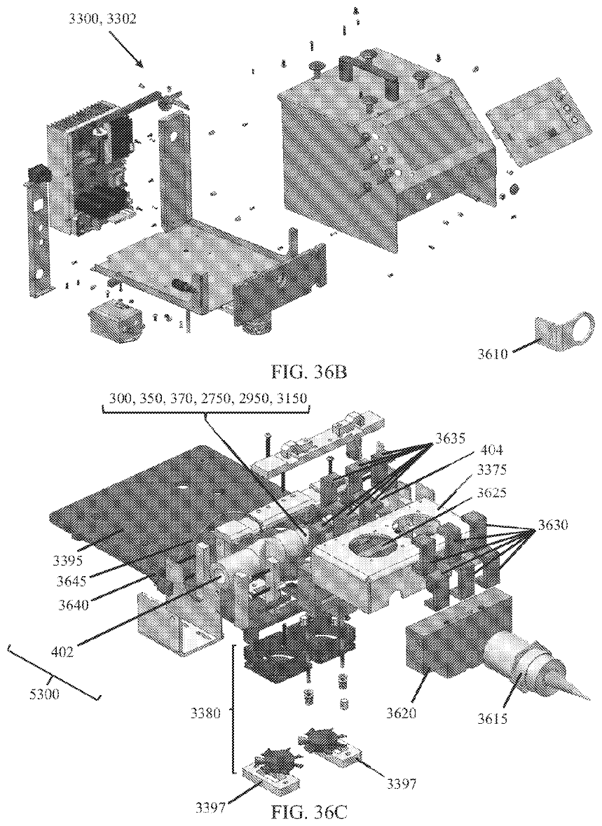

FIG. 36A is a perspective view of a main housing of the induction heating device 3302 of the third system 3300 according to an exemplary embodiment. The induction heating device 3302 may include a manifold plate 3605 for connecting the main housing with the cradle 5300 and other components.

FIG. 36B is an exploded perspective view of the main housing of the induction heating device 3302 of the third system 3300 according to an exemplary embodiment. An L-shaped clamp 3610 with a circular opening in one end may be provided to structurally support an infrared laser sensor (not shown) of the laser 3615 for temperature measurement (see, FIG. 36C).

FIG. 36C is an exploded perspective view of the third cradle 5300, the conduits 402, 404, the insert 300, 350, 370, 2750, 2950, 3150, inductive heating coils 3630, 3635, a laser 3615 for temperature measurement, a pair of the induction blocks 3620, 3625, the thermocouples 3640 (including the wiring 3645 for the thermocouples 3640), the pair of cooling fans 3380, the protective shield 3375, the base platform 3395, and a pair of the clamp bases 3397 of the third system 3300 according to an exemplary embodiment.

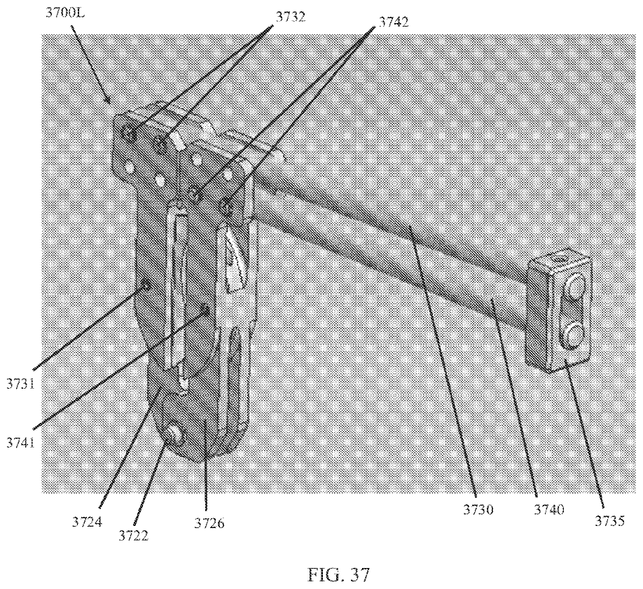

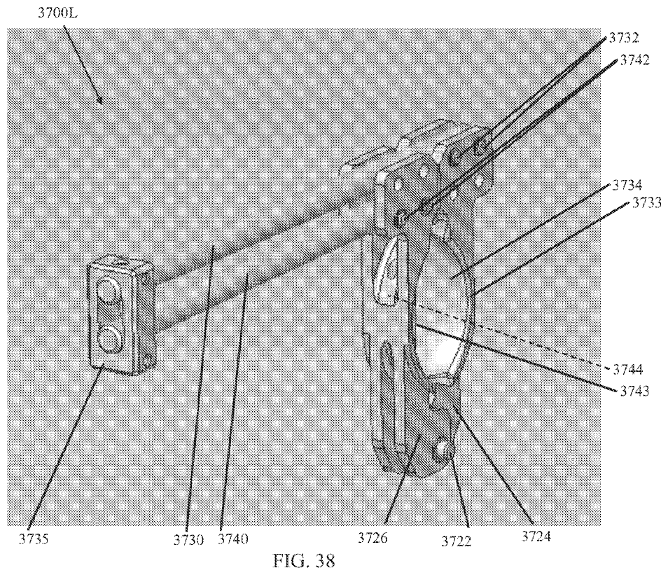

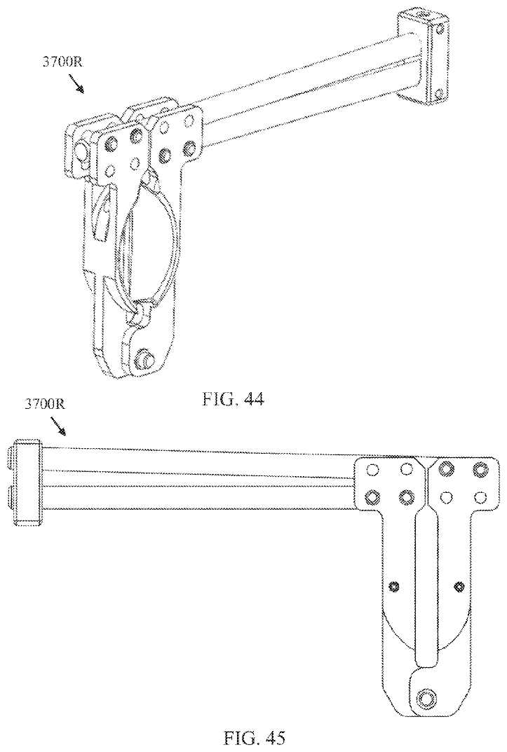

FIGS. 37-50 disclose details of the third clamps 3700. FIGS. 37-43 disclose details of a left-hand-side clamp 3700L of the pair of third clamps 3700. FIGS. 44-50 disclose details of a right-hand-side one 3700R of the pair of third clamps 3700. The left-side clamp 3700L and the right-side clamp 3700R are structurally similar, and oriented differently, as appropriate to their function.

FIG. 37 is a lateral-facing side perspective view of the left-hand-side clamp 3700L of the pair of third clamps 3700 in a closed position according to an exemplary embodiment. The left-hand-side clamp 3700L may include a first conduit engaging armature 3724 and a second conduit engaging armature 3726 configured to be joined and to commonly pivot about a pin 3722. Conduit engagement surfaces (described below) may be connected to the first conduit engaging armature 3724 and the second conduit engaging armature 3726 via pins 3731, 3741, respectively. A first arm 3730 may be connected to an open end of the first conduit engaging armature 3724 via pins 3732. A second arm 3740 may be connected to an open end of the second conduit engaging armature 3726 via pins 3742. When the left-hand-side clamp 3700L is in the closed position, the first arm 3730 and the second arm 3740 may be biased apart so as to be slightly non-parallel with respect to each other. A tension block 3735 may be configured to slip over ends of the first arm 3730 and the second arm 3740 to tighten and/or secure a clamping force of the left-hand-side clamp 3700L on the one or more conduits 402, 404.

FIG. 38 is a medial-facing side perspective view of the left-hand-side clamp 3700L of the pair of third clamps 3700 in the closed position according to an exemplary embodiment. The conduit engagement surfaces may be integrated into the first conduit engaging armature 3724 and the second conduit engaging armature 3726 (not shown). The conduit engagement surfaces may be provided as separate components and connected to the first conduit engaging armature 3724 and the second conduit engaging armature 3726 via the pins 3731, 3741, as shown.

The conduit engagement surfaces may include a posterior transition surface 3733 and a primary posterior engagement surface 3734. The posterior transition surface 3733 may be a curved arc providing a smooth transition from a curved surface of the posterior transition surface 3733 to a relatively flat surface of the first conduit engaging armature 3724. The conduit engagement surfaces may include an anterior transition surface 3743 and a primary anterior engagement surface 3744. The anterior transition surface 3743 may be a curved arc providing a smooth transition from a curved surface of the anterior transition surface 3743 to a relatively flat surface of the second conduit engaging armature 3726.

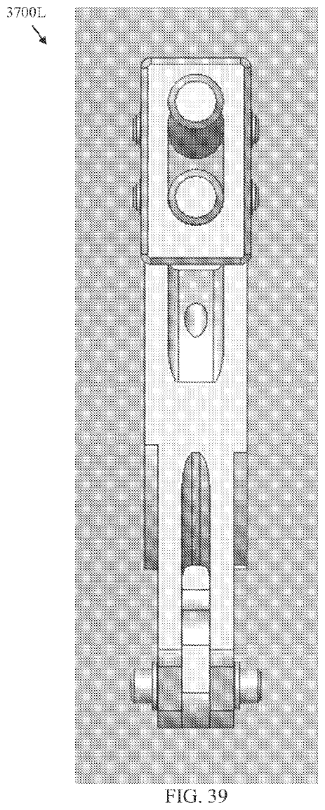

FIG. 39 is an anterior side view of the left-hand-side clamp 3700L of the pair of third clamps 3700 in the closed position according to an exemplary embodiment.

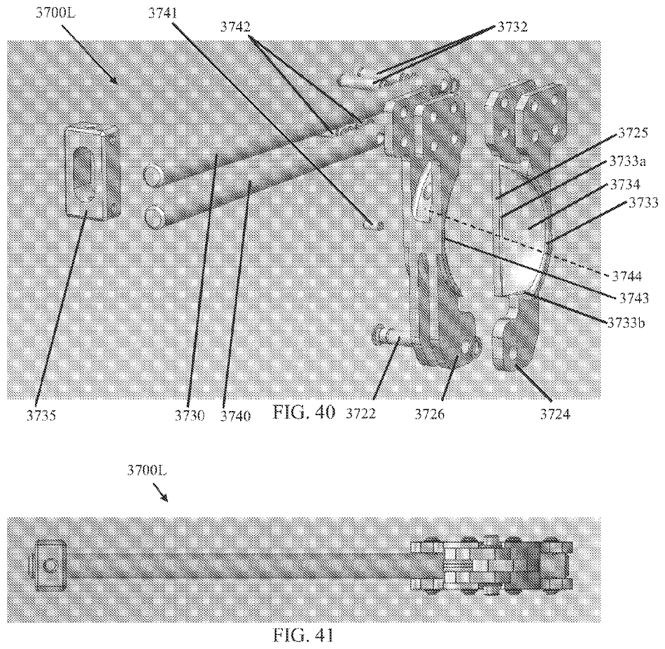

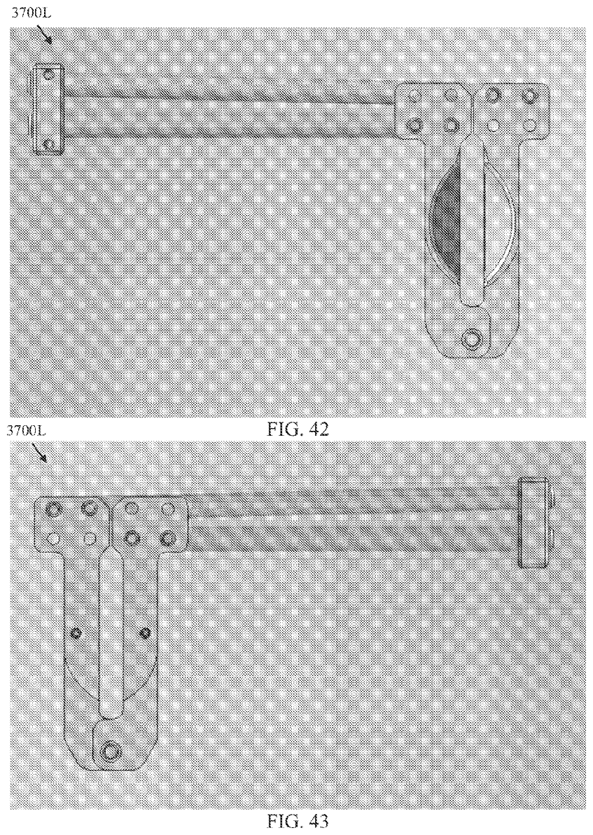

FIG. 40 is an exploded medial-facing side perspective view of the left-hand-side clamp 3700L of the pair of third clamps 3700 in the closed position according to an exemplary embodiment. The conduit engagement surfaces may include a posterior transition surface 3733a and a secondary posterior engagement surface 3725. The posterior transition surface 3733a may be a tapered and curved arc providing a smooth transition from a curved surface of the posterior transition surface 3733a to a relatively flat surface of the secondary posterior engagement surface 3725. The conduit engagement surfaces may include a pair of secondary posterior transition surfaces 3733b on either side of the primary posterior engagement surface 3734. Each of the pair of secondary posterior transition surfaces 3733b may have a triangular arced shape configured to provide a smooth transition from the curved surface of the primary posterior engagement surface 3734 and the relatively flat surface of the secondary posterior engagement surface 3725. The secondary posterior engagement surface 3725 may have three regions bounding top, lateral and bottom sides of the primary posterior engagement surface 3734.

The conduit engagement surfaces are not limited to those illustrated and may include any suitable shape. The conduit engagement surfaces may be configured to provide a maximum clamping force at the secondary posterior engagement surface 3725. Other portions of the conduit engagement surfaces may provide relatively less clamping force but ensure contact of the clamp left-hand-side clamp 3700L with the conduit in the Zone C as described above with respect to FIGS. 14 and 15. The superior performance of such conduit engagement surfaces is demonstrated, for example, by FIG. 16B. In summary, by using clamps with conduit engagement surfaces as described hereinabove, a critical temperature for sterilization (e.g., 160° C. (320° F.)) may be achieved relatively quickly (e.g., in about 120 seconds) compared to configurations omitting such conduit engagement surfaces.

Please note, the conduit engagement surfaces of the first conduit engaging armature 3724 may be similar to the conduit engagement surfaces of the second conduit engaging armature 3726. Detailed descriptions are omitted for brevity.

FIG. 41 is a bottom side view of the left-hand-side clamp 3700L of the pair of third clamps 3700 in the closed position according to an exemplary embodiment.

FIG. 42 is a medial side view of the left-hand-side clamp 3700L of the pair of third clamps 3700 in the closed position according to an exemplary embodiment.

FIG. 43 is a lateral side view of the left-hand-side clamp 3700L of the pair of third clamps 3700 in the closed position according to an exemplary embodiment.

As shown in FIGS. 44-50, the right-hand-side clamp 3700R may be a mirror image of the left-hand-side clamp 3700L. The right-hand-side clamp 3700R may include some or all the above-referenced features of the left-hand-side clamp 3700L. Detailed descriptions are omitted for brevity.

FIG. 44 is a medial-facing side perspective view of the right-hand-side clamp 3700R of the pair of third clamps 3700 in a closed position according to an exemplary embodiment.

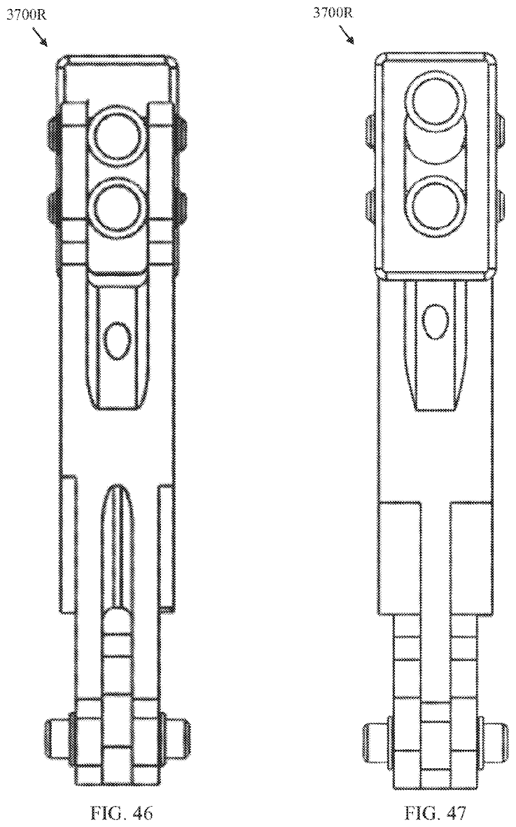

FIG. 45 is a lateral side view of the right-hand-side clamp 3700R of the pair of third clamps 3700 in the closed position according to an exemplary embodiment.

FIG. 46 is a posterior side view of the right-hand-side clamp 3700R of the pair of third clamps 3700 in the closed position according to an exemplary embodiment.

FIG. 47 is an anterior side view of the right-hand-side clamp 3700R of the pair of third clamps 3700 in the closed position according to an exemplary embodiment.

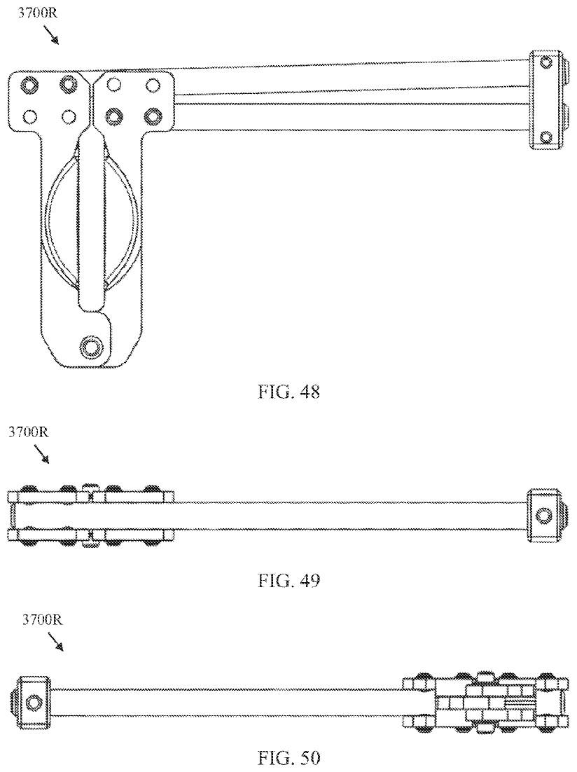

FIG. 48 is a medial side view of the right-hand-side clamp 3700R of the pair of third clamps 3700 in the closed position according to an exemplary embodiment.

FIG. 49 is a top side view of the right-hand-side clamp 3700R of the pair of third clamps 3700 in the closed position according to an exemplary embodiment.

FIG. 50 is a bottom side view of the right-hand-side clamp 3700R of the pair of third clamps 3700 in the closed position according to an exemplary embodiment.

FIG. 51 is a front perspective view of the third system 3300 for making a sterile connection between at least one conduit (e.g., 402, 404) and an insert 300, 350, 370, 2750, 2950, 3150 including the induction heating device 3302, the third cradle 5300, and the pair of third clamps 3700 according to an exemplary embodiment. FIG. 51 discloses exemplary features of the touch screen display 3325. The touch screen display 3325 may include one or more of the following features in any suitable combination: a home button 5105, a status indicator 5110 (which may be configured to display a stage of the sterilization process, e.g., “SOAK”), a page advance button 5115, a power indicator 5120 (e.g., power expressed as a percentage of maximum and wattage, e.g., “POWER”, “5%” and “98/2000 W”), a current indicator 5125 (e.g., current expressed as a percentage of maximum and amperage, e.g., “CURRENT”, “29%” and “2.9/10 A”), a voltage indicator 5130 (e.g., voltage expressed as a percentage of maximum and voltage, e.g., “VOLTAGE”, “14%” and “34/250 V”), a conduit size indicator 5135 (e.g., “¾”, which, in this exemplary embodiment, refers to a conduit having a diameter of about 0.75 inches (about 1.905 centimeters)), a clamp 1 temperature indicator 5140 (e.g., “CLAMP 1” and “183° C. (361.4° F.)”), a clamp 2 temperature indicator 5145 (e.g., “CLAMP 2” and “195° C. (383° F.)”), a main sensor temperature indicator 5150 (e.g., “MAIN SENSOR” and “170° C. (338° F.)”), a set point temperature indicator 5155 (e.g., “MAIN SP” and “170° C. (338° F.)”, which is an exemplary set point temperature of the insert 300, 350, 370, 2750, 2950, 3150), a maximum clamp temperature indicator (e.g., “MAX CLAMP” and “350° C. (662° F.)”), a frequency indicator 5165 (e.g., “87 kHz”), a more detail button 5170, and a timer display (e.g., 00:57 min”). The touch screen display 3325 may be configured to display less or more information on different screens and/or depending on the operation mode the third system 3300.

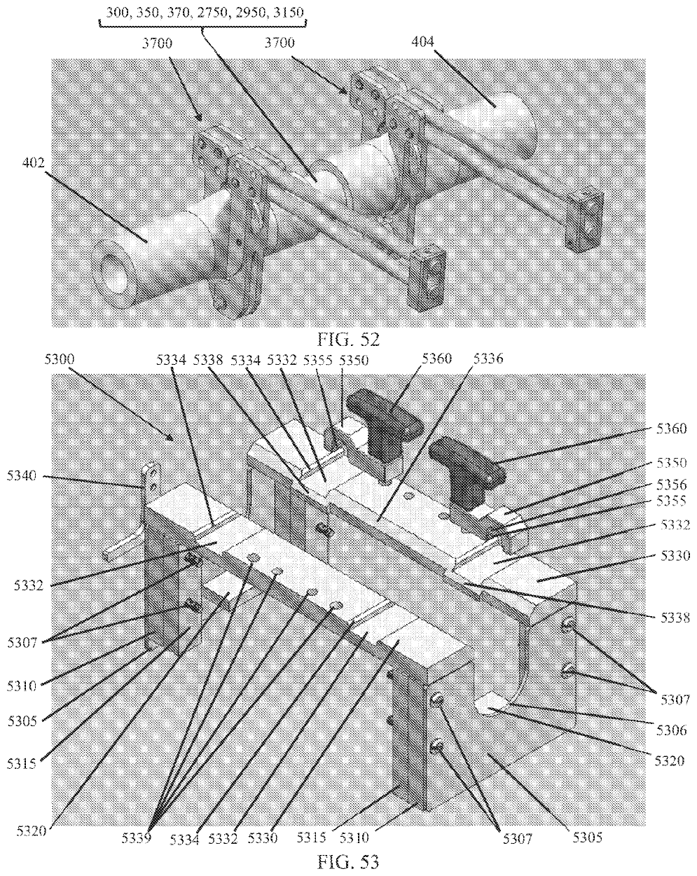

FIG. 52 is a perspective view of the pair of third clamps 3700 engaged with a pair of conduits 402, 404, which are connected with an insert 300, 350, 370, 2750, 2950, 3150 according to an exemplary embodiment. Please note, on medial sides of the clamps 3700, the conduit engagement surfaces of the clamps 3700 are in snug yet gradual engagement with the conduits 402, 404. Whereas, on lateral sides of the clamps 3700, the conduit engagement surfaces of the clamps 3700 are in a relatively tight constrictive clamped or pinching engagement with the conduits 402, 404.

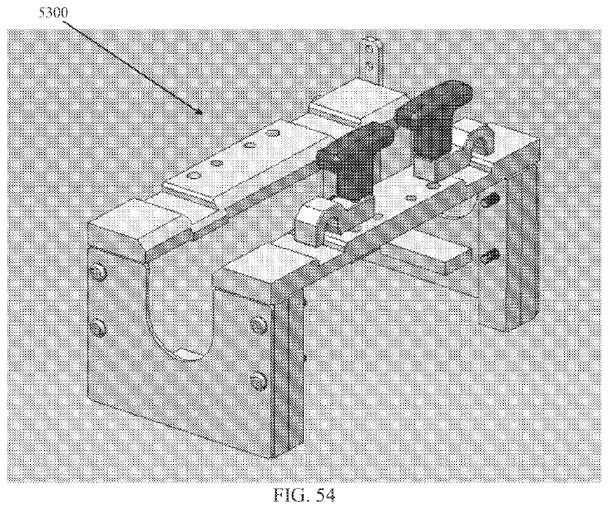

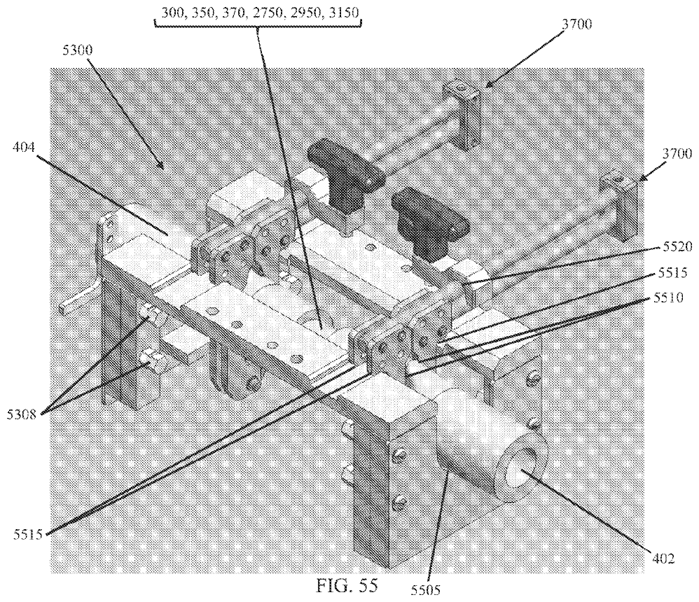

FIG. 53 is an anterior perspective view of the third cradle 5300 according to an exemplary embodiment. The third cradle 5300 may include one or more of the following features in any suitable combination: a pair of end plates 5305, each with a U-shaped opening 5306, a plurality (e.g., 8) of attachment bolts 5307, a plurality (e.g., 4) lateral blocks 5310, a plurality (e.g., 4) of medial blocks 5315, a pair of medial blocks 5320, a pair of T-bars 5330, each with a pair of clamp engagement grooves 5332, each groove 5332 with chamfers 5334 on either side, each T-bar 5330 with a chamfer 5336 and a pair of protrusions 5338 facing the conduit 402, 404, each T-bar 5330 with a plurality (e.g., 4) of openings 5339 for facilitating connection with other structures (including the spring-loaded bolts 5710 described elsewhere), an L-shaped lever arm 5340, which may be operatively connected to the third system 3300 to indicate, by its position relative to a sensor (not shown), whether the safety shield 575 or the protective cover 3375 or the like is in an open position or closed position, a pair of secondary clamps 5350 for engagement with the third clamps 3700, each secondary clamp 5350 having an inverted U-shaped opening 5356 with a chamfer 5355 on either side for engagement with arms of the clamps 3700, and a pair of T-shaped handles 5360 for lifting the secondary clamps 5350.

FIG. 53 is a posterior perspective view of the third cradle 5300 according to an exemplary embodiment.

FIG. 54 is an anterior perspective view of the third cradle 5300 according to an exemplary embodiment.

FIG. 55 is a posterior perspective view of the third cradle 5300 engaged with the pair of conduits 402, 404 and the pair of third clamps 3700 (also, the insert 300, 350, 370, 2750, 2950, 3150 between the pair of conduits 402, 404) according to an exemplary embodiment. The clamps 3700 and the third cradle 5300 are configured to provide a first support region 5505 for each of the conduits 402, 404 (one first support region 5505 is shown), a pair of second support regions 5510 between each of the third clamps 3700 and each of the conduits 402, 404, four third support regions 5515 between the cradle 5300 and each of the third clamps 3700, and a fourth support region 5520 between each of the third clamps 3700 and each of the secondary clamps 5350. The third cradle 5300 may include hex nuts 5308 for tightening the attachment bolts 5307.

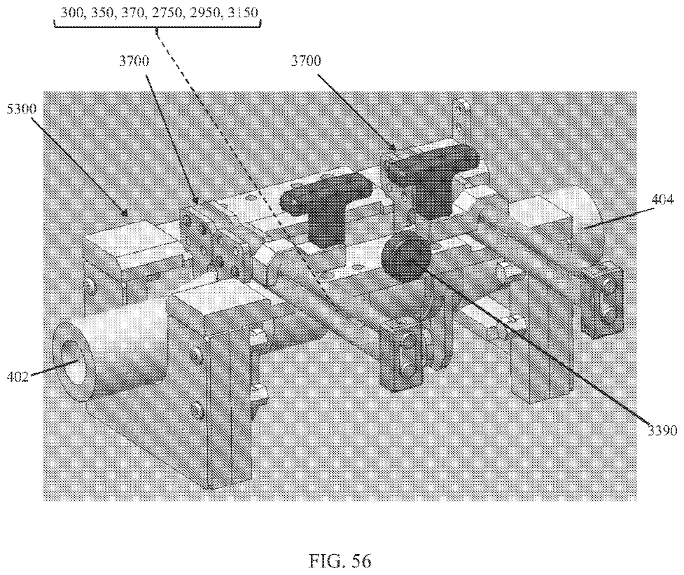

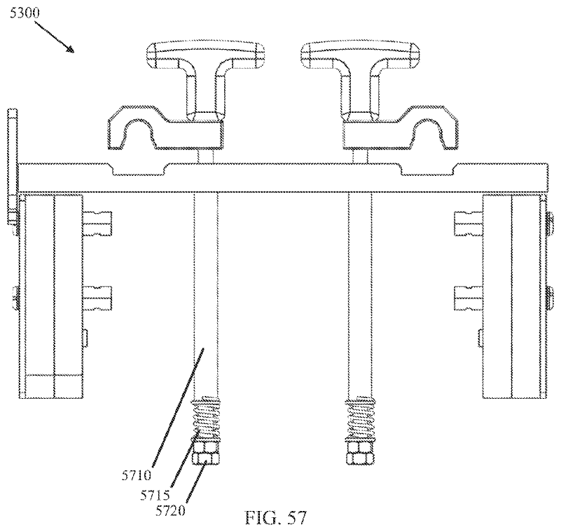

FIG. 56 is an anterior perspective view of the third cradle 5300 engaged with the pair of conduits 402, 404 and the pair of third clamps 3700 (also, the insert 300, 350, 370, 2750, 2950, 3150 between the pair of conduits 402, 404) according to an exemplary embodiment. The knob 3390 is illustrated in FIG. 56 merely to help correlate the view of FIG. 56 with other views (the knob 3390 does not form part of the assembly, the cradle, the clamps, or the like).

FIG. 57 is a posterior side view of the third cradle 5300 including bolts 5710, springs 5715 for biasing the bolts 5710, and hex nuts 5720 for tightening the bolts 5710 according to an exemplary embodiment.

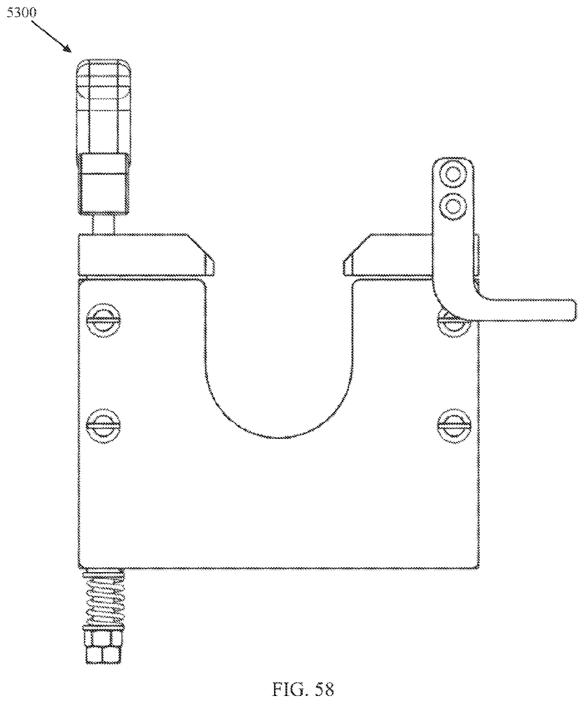

FIG. 58 is a right-hand side view of the third cradle 5300 including spring loaded bolts and hex nuts according to an exemplary embodiment.

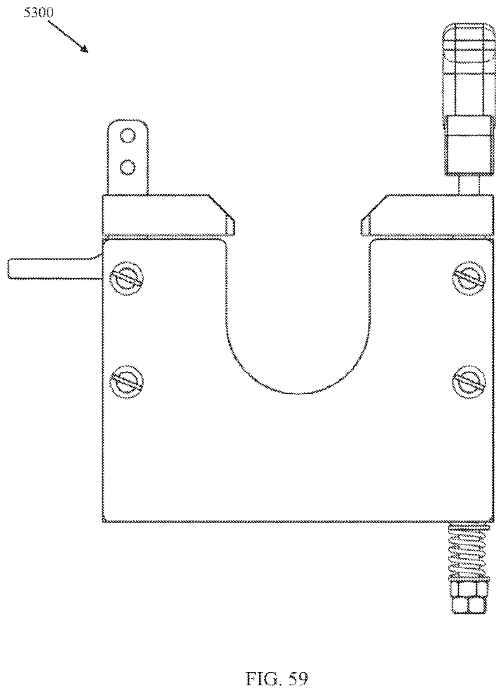

FIG. 59 is a left-hand side view of the third cradle 5300 including spring loaded bolts and hex nuts according to an exemplary embodiment.

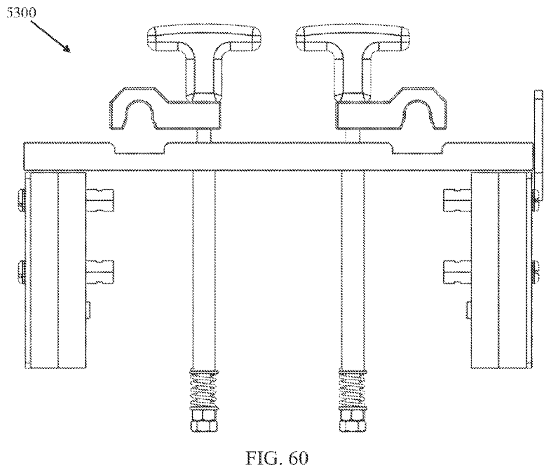

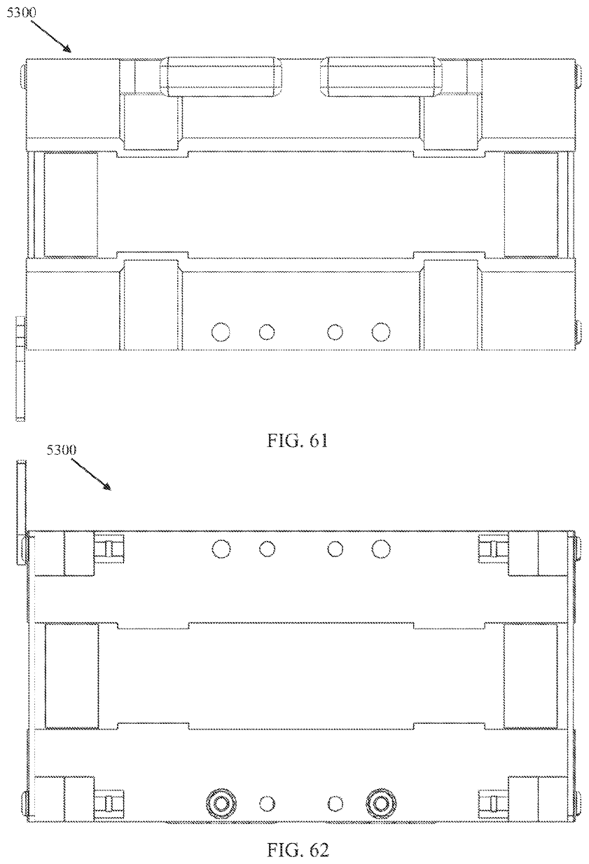

FIG. 60 is an anterior side view of the third cradle 5300 including spring loaded bolts and hex nuts according to an exemplary embodiment.

FIG. 61 is a top side view of the third cradle 5300 including spring loaded bolts and hex nuts according to an exemplary embodiment.

FIG. 62 is a bottom side view of the third cradle 5300 including spring loaded bolts and hex nuts according to an exemplary embodiment.

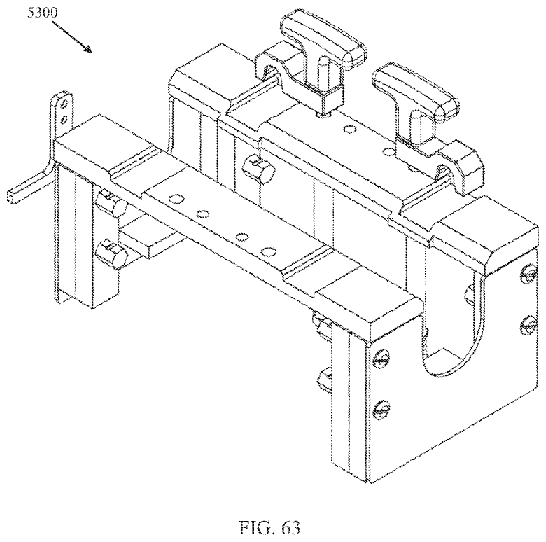

FIG. 63 is a posterior perspective view of the third cradle 5300 including spring loaded bolts and hex nuts according to an exemplary embodiment.

### Terminology

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Although at least one exemplary embodiment is described as using a plurality of units to perform the exemplary process, it is understood that the exemplary processes may also be performed by one or plurality of modules.

The use of the terms “first”, “second”, “third” and so on, herein, are provided to identify various structures, dimensions or operations, without describing any order, and the structures, dimensions or operations may be executed in a different order from the stated order unless a specific order is definitely specified in the context.

Approximating language, as used herein throughout the specification and claims, may be applied to modify any quantitative representation that could permissibly vary without resulting in a change in the basic function to which it is related. Accordingly, a value modified by a term or terms, such as “about” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein may be embodied in systems, apparatus, methods, and/or articles depending on the desired configuration. The embodiments set forth in the foregoing description do not represent all embodiments consistent with the subject matter described herein. Instead, they are merely some examples consistent with aspects related to the described subject matter. Although a few variations have been described in detail above, other modifications or additions are possible. In particular, further features and/or variations may be provided in addition to those set forth herein. For example, the embodiments described above may be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other embodiments may be within the scope of the following claims.

What is claimed is:

1. An insert for connecting a conduit having an outer surface and an inner surface, the insert comprising:
a body having an inner surface and an outer surface, the body configured to connect with the conduit having the outer surface and the inner surface,
wherein the outer surface of the body of the insert is configured to engage with the inner surface of the conduit,
wherein the body is configured for inductive heating of a connection of the insert and the conduit to a sterilizing temperature,
wherein the body is configured for inductive heating for a duration of time, and wherein an interior of the connection of the insert and the conduit is sterilized as a result of the inductive heating.

2. The insert of claim 1, wherein the sterilizing temperature is between about 160° C. and about 350° C., and the duration of time is between about 60 seconds and about 300 seconds.

3.-7. (canceled)

8. A system comprising the insert of claim 1 and a clamp configured to transmit inductive heat to the conduit.

9.-16. (canceled)

17. A method for connecting a conduit with an insert, the method comprising:
clamping a conduit with a clamp;
cutting a portion of the conduit leaving an extending portion;
inserting an end of the insert into the extending portion;
securing the extending portion to the insert with a compression device;
electrically connecting an inductive heating device to the insert;
heating the insert with energy from the inductive heating device;
maintaining a predetermined temperature for a duration of time in order to form a sterile connection of an interior of the conduit and the insert;
soaking the sterile connection;
cooling the sterile connection; and
removing the clamp from the sterile connection.

18. (canceled)

19. The method of claim 17, wherein the predetermined temperature is between about 160° C. and about 350° C.

20. (canceled)

21. The method of claim 17, wherein electrically connecting the inductive heating device to the insert includes electrically connecting the inductive heating device to the clamp, and wherein heating the insert with energy from the inductive heating device includes heating the clamp with energy from the inductive heating device.

22. (canceled)

23. A method of connecting a conduit with an insert, wherein a device is provided, the device having at least one processor and a memory storing at least one program for execution by the at least one processor, the at least one program including instructions, which, when executed by the at least one processor cause the at least one processor to perform operations comprising:
transmitting instructions to an inductive heating device to transmit heat energy to the insert from the inductive heating device;
determining a temperature of at least one sensor within the conduit and/or the insert; and
maintaining the determined temperature equal to or above a predetermined temperature for a duration of time in order to form a sterile connection of an interior of the conduit and the insert.

24. The method of claim 23, wherein the predetermined temperature is between about 160° C. and about 350° C.

25. A system for connecting a conduit with an insert, the system comprising:
a device having at least one processor and a memory storing at least one program for execution by the at least one processor, the at least one program including instructions, when, executed by the at least one processor cause the at least one processor to perform operations comprising:
transmitting instructions to an inductive heating device to transmit heat energy to the insert from the inductive heating device;
determining a temperature of at least one sensor within the conduit and/or the insert; and
maintaining the determined temperature equal to or above a predetermined temperature for a duration of time in order to form a sterile connection of an interior of the conduit and the insert.

26. The system of claim 25, wherein the predetermined temperature is between about 160° C. and about 350° C.

27. A non-transitory computer-readable storage medium storing at least one program for connecting a conduit with an insert, the at least one program for execution by at least one processor and a memory storing the at least one program, the at least one program including instructions, when, executed by the at least one processor cause the at least one processor to perform operations comprising:
transmitting instructions to an inductive heating device to transmit heat energy to the insert from the inductive heating device;
determining a temperature of at least one sensor within the conduit and/or the insert; and
maintaining the determined temperature equal to or above a predetermined temperature for a duration of time in order to form a sterile connection of an interior of the conduit and the insert.

28. The non-transitory computer-readable storage medium of claim 27, wherein the predetermined temperature is between about 160° C. and about 350° C.

29. A clamp for transmitting inductive heat to a conduit, the clamp comprising:
carbon steel; and
a base and a collar extending beyond the base,
wherein the collar is configured to make contact with a portion of the conduit in a critical zone located between a clamp zone directly adjacent to a clamp attached to the conduit and an insulated zone formed by an overlap of an insert and the conduit.

30. (canceled)

31. The clamp of claim 29, wherein the collar has an inwardly facing surface configured to substantially conform with an outer surface of the clamped conduit.

32. (canceled)

33. An apparatus for indirectly supporting an insert and making direct contact with a clamp and for delivering energy from an external energy source to the insert and the clamp in order to generate inductive heat in the insert and the clamp, the apparatus comprising:
conductive material;
a main groove in a top of a body of the apparatus;
at least one peripheral block;
a central groove in the at least one peripheral block, the central groove aligned with the main groove;
at least one central block; and
a peripheral groove formed between the at least one peripheral block and the at least one central block,
wherein the apparatus is configured to concentrate inductive heat within one or more of the main groove, the central groove and/or the peripheral groove.

34. The apparatus of claim 33, wherein the apparatus is configured to promote development of a predetermined temperature of an interior of the insert, the clamp, and the conduit, the predetermined temperature being between about 160° C. and about 350° C.

35. (canceled)

36. (canceled)

37. An apparatus for indirectly supporting an insert and making direct contact with a clamp and for positioning the insert and the clamp so that energy is delivered from an external energy source to the insert and the clamp in order to generate inductive heat in the insert and the clamp, the apparatus comprising:
a conduit support surface;
an accommodating space for accommodating a conduit, the insert and the clamp;
a first clamp support surface; and
a second clamp support surface,
wherein the apparatus is configured to concentrate inductive heat within the accommodating space.

38. The apparatus of claim 37, wherein the apparatus is configured to promote development of a predetermined temperature of an interior of the insert, the clamp, and the conduit, the predetermined temperature being between about 160° C. and about 350° C.

39. The apparatus of claim 37 further configured to accommodate or hold a conduit, the insert, and the clamp during the generation of the inductive heat.

40.-51. (canceled)

52. A clamp for indirectly supporting an insert and for making direct contact with a clamp and for delivering energy from an external energy source to the insert and the clamp in order to generate inductive heat in the insert and the clamp, the clamp comprising:
a collar including conduit engagement surfaces,
wherein the conduit engagement surfaces include a posterior transition surface and a secondary posterior engagement surface,
wherein the posterior transition surface is a tapered and curved arc providing a smooth transition from a curved surface of the posterior transition surface to a relatively flat surface of the secondary posterior engagement surface,
wherein the conduit engagement surfaces include a pair of secondary posterior transition surfaces on either side of the primary posterior engagement surface, and
wherein each of the pair of secondary posterior transition surfaces has a triangular arced shape configured to provide a smooth transition from the curved surface of the primary posterior engagement surface and the relatively flat surface of the secondary posterior engagement surface.
